(19) United States
(12) Patent Application Publication
(10) Pub. No.: US 2025/0237589 A1
(43) Pub. Date: Jul. 24, 2025

(54) SYSTEMS AND METHODS FOR A SELF-VERIFYING CAPACITIVE SENSOR SYSTEM

(71) Applicant: Sensia Netherlands B.V., Rotterdam (NL)

(72) Inventors: Gregor James Brown, Prestwick (GB); Stuart Fraser Wright, Battle (GB); Albert Hoefel, Sugar Land, TX (US)

(73) Assignee: Sensia Netherlands B.V., Rotterdam (NL)

(21) Appl. No.: 18/416,250

(22) Filed: Jan. 18, 2024

(51) Int. Cl. G01N 11/02; G01N 11/00; G01N 33/28

(52) U.S. Cl. CPC G01N 11/02; G01N 33/2847; G01N 2011/0066

(57) ABSTRACT

A system includes a capacitive sensor and a processor. The capacitive sensor is configured to measure a first capacitance along a first path through a fluid and a second capacitance along a second path through the fluid. The processor is configured to receive sensor data from the capacitive sensor corresponding to the first capacitance and the second capacitance. The processor is also configured to determine a first value of a fluid property of the fluid based on the first capacitance. The processor is also configured to determine a second value of the fluid property of the fluid based on the second capacitance. The processor is also configured to operate a display device to provide the variation to a user in response to determining that a variation between the first value of the fluid property and the second value of the fluid property is above a predetermined threshold.

### BACKGROUND

The present application relates generally to the field of calibration of sensors. More specifically, the present application relates to the field of calibration of capacitive sensors.

### SUMMARY OF THE INVENTION

One embodiment of the invention relates to a system including a capacitive sensor and a processor, according to some embodiments. In some embodiments, the capacitive sensor is configured to measure a first capacitance along a first path through a fluid and a second capacitance along a second path through the fluid. In some embodiments, the first path is different from the second path. In some embodiments, the processor is configured to receive sensor data from the capacitive sensor corresponding to the first capacitance and the second capacitance. In some embodiments, the processor is configured to determine a first value of a fluid property of the fluid based on the first capacitance. In some embodiments, the processor is configured to determine a second value of the fluid property of the fluid based on the second capacitance. In some embodiments, the processor is configured to operate a display device to provide the variation to a user in response to determining that a variation between the first value of the fluid property and the second value of the fluid property is above a predetermined threshold.

In some embodiments, the capacitive sensor is further configured to measure a third capacitance along a third path through the fluid. In some embodiments, the third path is different from the first path and the second path. In some embodiments, the processor is configured to receive the sensor data from the capacitive sensor corresponding to the third capacitance. In some embodiments, the processor is configured to determine a third value of the fluid property of the fluid based on the third capacitance. In some embodiments, the processor is configured to model the first value of the fluid property with the second value of the fluid property and the third value of the fluid property to determine a sensor error associated with at least one of the first value of the fluid property, the second value of the fluid property, or the third value of the fluid property. In some embodiments, the processor is configured to operate the display device to provide the sensor error to the user in response to determining that the sensor error is above a predetermined error threshold.

In some embodiments, the processor is further configured to calibrate the capacitive sensor to reduce the sensor error in response to determining that the sensor error is above the predetermined error threshold.

In some embodiments, the capacitive sensor includes a first electrode, a second electrode, a third electrode, and a fourth electrode. In some embodiments, the first path is between (i) the first electrode and the third electrode and (ii) the second electrode and the fourth electrode. In some embodiments, the second path is between the first electrode and the third electrode. In some embodiments, the third path is between the second electrode and the fourth electrode.

In some embodiments, the fluid is in a conduit. In some embodiments, the first electrode and the third electrode are configured to be positioned on a first side of the conduit. In some embodiments, the second electrode and the fourth electrode are configured to be positioned on a second side of the conduit, the second side opposite the first side.

In some embodiments, the capacitive sensor includes a shield positioned between (i) the first electrode and the third electrode and (ii) the second electrode and the fourth electrode. In some embodiments, the shield is configured to partially block electric flux between (i) the first electrode and the third electrode and (ii) the second electrode and the fourth electrode.

In some embodiments, the sensor error is associated with a deposit of a material on a surface of the capacitive sensor or an erosion of the surface of the capacitive sensor due to the fluid.

In some embodiments, the processor is configured to determine a location of (i) the deposit of the material on the capacitive sensor or (ii) the erosion of the capacitive sensor based on the sensor error. In some embodiments, the processor is configured to operate the display device to provide the location of (i) the deposit of the material or (ii) the erosion of the capacitive sensor to the user.

In some embodiments, the fluid property is a permittivity of the fluid. In some embodiments, the processor is further configured to determine a water fraction of the fluid based on the permittivity of the fluid.

Another implementation of the present disclosure is a system including a capacitive sensor and a processor, according to some embodiments. In some embodiments, the capacitive sensor includes a plurality of electrode pairs. In some embodiments, each of the electrode pairs configured to measure a capacitance through a fluid between a first electrode and a second electrode of each of the electrode pairs. In some embodiments, the processor is configured to receive sensor data from the capacitive sensor corresponding to a first capacitance through a fluid parcel of the fluid at a first time from a first of the electrode pairs and a second capacitance through the fluid parcel of the fluid at a second time from a second of the electrode pairs. In some embodiments, the processor is configured to determine a first value of a fluid property of the fluid based on the first capacitance. In some embodiments, the processor is configured to determine a second value of the fluid property of the fluid based on the second capacitance, the second value substantially equal to the first value. In some embodiments, the processor is configured to determine a velocity of the fluid based on the first time and the second time. In some embodiments, the processor is configured to operate a display device to provide the velocity of the fluid to a user.

In some embodiments, the processor is configured to determine a third time when the fluid parcel of the fluid will be positioned proximate a third of the electrode pairs of the capacitive sensor. In some embodiments, the processor is configured to receive the sensor data from the capacitive sensor corresponding to a third capacitance through the fluid parcel of the fluid at the third time from a third of the electrode pairs. In some embodiments, the processor is configured to determine a third value of the fluid property of the fluid based on the third capacitance. In some embodiments, the processor is configured to operate the display device to provide the variation to the user in response to determining that a variation between the first value of the fluid property and the third value of the fluid property is above a predetermined threshold.

In some embodiments, the processor is configured to receive the sensor data from the capacitive sensor corresponding to a fourth capacitance through the fluid parcel from the first electrode of the first of the electrode pairs and the first electrode of the second of the electrode pairs. In some embodiments, the processor is configured to determine a fourth value of the fluid property of the fluid based on the fourth capacitance. In some embodiments, the processor is configured to model the fourth value of the fluid property with the first value of the fluid property, the second value of the fluid property, and the third value of the fluid property to determine a sensor error associated with at least one of the first of the electrode pairs, the second of the electrode pairs, or the third of the electrode pairs. In some embodiments, the processor is further configured to operate the display device to provide the sensor error to the user in response to determining that the sensor error is above a predetermined error threshold.

In some embodiments, the processor is configured to calibrate the capacitive sensor to reduce the sensor error in response to determining that the sensor error is above the predetermined error threshold.

In some embodiments, the fluid parcel of the fluid is a first fluid parcel of the fluid. In some embodiments, the velocity is an initial velocity. In some embodiments, the processor is configured to receive the sensor data corresponding to a first calibrated capacitance through a second fluid parcel of the fluid measured at a fourth time from the first of the electrode pairs and a second calibrated capacitance through the second fluid parcel measured at a fifth time from the second of the electrode pairs. In some embodiments, the processor is configured to determine a first calibrated value of the fluid property based on the first calibrated capacitance. In some embodiments, the processor is configured to determine a second calibrated value of the fluid property based on the second calibrated capacitance. In some embodiments, the second calibrated value substantially equal to the first calibrated value. In some embodiments, the processor is configured to determine a calibrated velocity of the fluid based the fourth time and the fifth time. In some embodiments, the processor is configured to operate the display device to provide the calibrated velocity of the fluid to the user.

In some embodiments, the sensor error is associated with a deposit of a material on a surface of the capacitive sensor or an erosion of the surface of the capacitive sensor due to the fluid.

In some embodiments, the processor is configured to determine a location of (i) the deposit of the material on the capacitive sensor or (ii) the erosion of the capacitive sensor based on the sensor error. In some embodiments, the processor is configured to operate the display device to provide the location of (i) the deposit of the material or (ii) the erosion of the capacitive sensor to the user.

In some embodiments, the fluid property is a permittivity of the fluid. In some embodiments, the processor is further configured to determine a water fraction of the fluid based on the permittivity of the fluid.

Another implementation of the present disclosure is a method, according to some embodiments. In some embodiments, the method includes receiving sensor data from a capacitive sensor corresponding to a first capacitance though a fluid along a first path and corresponding to a second capacitance through the fluid along a second path. In some embodiments, the method includes determining a first value of a fluid property of the fluid based on the first capacitance. In some embodiments, the method includes determining a second value of the fluid property of the fluid based on the second capacitance. In some embodiments, the method includes operating a display device to provide the variation to a user in response to determining that a variation between the first value of the fluid property and the second value of the fluid property is above a predetermined threshold.

In some embodiments, the method includes receiving the sensor data from the capacitive sensor corresponding to a third capacitance through the fluid along a third path. In some embodiments, the method includes determining a third value of the fluid property of the fluid based on the third capacitance. In some embodiments, the method includes modeling the first value of the fluid property with the second value of the fluid property and the third value of the fluid property to determine a sensor error associated with the capacitive sensor and at least one of the first value of the fluid property, the second value of the fluid property, or the third value of the fluid property. In some embodiments, the method includes operating the display device to provide the sensor error to the user in response to determining that the sensor error is above a predetermined error threshold.

In some embodiments, the method includes calibrating the capacitive sensor to reduce the sensor error in response to determining that the sensor error is above the predetermined error threshold.

In some embodiments, the fluid property is the permittivity of the fluid. In some embodiments, the method includes determining a water fraction of the fluid based on the permittivity of the fluid.

### BRIEF DESCRIPTION OF THE DRAWINGS

The disclosure will become more fully understood from the following detailed description, taken in conjunction with the accompanying figures, wherein like reference numerals refer to like elements, in which:

FIG. 1 is a schematic diagram of a system for a pipe system, according to some embodiments.

FIG. 2 is a block diagram of a control system of the pipe system of FIG. 1, according to some embodiments.

FIG. 3 is a cross section view of an example capacitive sensor of the pipe system of FIG. 1 taken along plane A-A, according to some embodiments.

FIG. 4 is a cross section view of configurations of an example capacitive sensor of the pipe system of FIG. 1 taken along plane A-A, according to some embodiments.

FIG. 5 is a cross section view of an example capacitive sensor of the pipe system of FIG. 1 taken along plane A-A, according to some embodiments.

FIG. 6 is a cross section view of an example electrode assembly of the capacitive sensor of FIG. 5 taken along plane B-B, according to some embodiments.

FIG. 7 is a perspective view of example electrode assemblies of the capacitive sensor of FIG. 5, according to some embodiments.

FIG. 8 is a cross section view of a first operational mode of the capacitive sensor of FIG. 5 taken along plane A-A, according to some embodiments.

FIG. 9 is a cross section view of a second operational mode of the capacitive sensor of FIG. 5 taken along plane A-A, according to some embodiments.

FIG. 10 is a cross section view of a third operational mode of the capacitive sensor of FIG. 5 taken along plane A-A, according to some embodiments.

FIG. 11 is a perspective cross section view of an example capacitive sensor of the pipe system of FIG. 1 taken along plane A-A, according to some embodiments.

FIG. 12 is a detail view of the cross section view of the capacitive sensor of FIG. 5, according to some embodiments.

FIG. 13 is a cross section view of another example capacitive sensor of the pipe system of FIG. 1 taken along plane A-A, according to some embodiments.

FIG. 14 is a graph displaying an effect of electrode wear on parallel capacitance measurements, according to some embodiments.

FIG. 15 is a graph displaying an effect of electrode wear on coplanar capacitance measurements, according to some embodiments.

FIG. 16 is a perspective view of another example capacitive sensor of the pipe system of FIG. 1, according to some embodiments.

FIG. 17 is a perspective view of electrode pairs of the capacitive sensor of FIG. 16, according to some embodiments.

FIG. 18 is a perspective view of another example capacitive sensor of the pipe system of FIG. 1, according to some embodiments.

FIG. 19 is a detail view of the capacitive sensor of FIG. 18, according to some embodiments.

FIG. 20 is a flow diagram of a process for validating a capacitive sensor, according to some embodiments.

FIG. 21 is a flow diagram of a process for determining a velocity of a fluid, according to some embodiments.

### DETAILED DESCRIPTION

Before turning to the FIGURES, which illustrate the exemplary embodiments in detail, it should be understood that the present application is not limited to the details or methodology set forth in the description or illustrated in the FIGURES. It should also be understood that the terminology is for the purpose of description only and should not be regarded as limiting.

### Overview

In the landscape of industrial operations (e.g., hydrocarbon transportation, hydrocarbon production, fluid storage, etc.), the deployment of sensors (e.g., composition sensors, temperature sensors, etc.) is critical for optimizing operations, ensuring safety, and maximizing efficiency. Sensors play a crucial role by providing real-time data and monitoring parameters throughout industrial processes. Specifically, capacitive sensors (e.g., capacitance sensors, capacitive sensor arrays, etc.) may be utilized for a variety of applications. For example, capacitive sensors may be used to measure a fluid level in a tank (e.g., a storage vessel, etc.), detecting leaks (e.g., spills, escaped fluid, etc.), sensing a flow through a fluid transportation network (e.g., a conduit, a pipeline, etc.), or measuring fluid permittivity of an constant enclosed fluid volume.

In order to monitor a fluid flowing through the fluid transportation network, some operators (e.g., energy companies, hydrocarbon transportation companies, midstream operators, etc.) may utilize the capacitive sensors to determine a composition of the fluid. The capacitive sensors may be utilized to determine a capacitance (e.g., an ability to store an electrical charge when subjected to a voltage, etc.) of the fluid, which may be utilized to determine a fluid permittivity (e.g., a dielectric constant, how well the fluid can be polarized by an electric field, etc.) of the fluid. The fluid permittivity can be a bulk parameter of the fluid that is dependent on the composition of the fluid. The fluid permittivity of the fluid may be used to determine a composition (e.g., a makeup, fractions of the fluid, etc.) a dispersion quality (e.g., uniformity, consistency, etc.), and/or a fluid property of the fluid based on known fluid properties (e.g., known fluid permittivity's of different fluids, etc.). For example, an operator may utilize a capacitance measurement from a capacitive sensor to determine a fluid permittivity of a mixture of oil and water flowing through a conduit. The operator may use the fluid permittivity of the mixture to determine a water composition of the mixture using known fluid composition properties such as the fluid permittivity of water and the fluid permittivity of oil. The operator may also use the fluid permittivity of the mixture to determine a dispersion of the water in the oil to determine a dispersion quality of the water in the oil.

However, in some instances components (e.g., water, oil, sand, etc.) of a fluid mixture may make up a low portion of the fluid mixture. Consequently, capacitance measurements taken by the capacitive sensors of the fluid mixture may be affected (e.g., skewed, have error introduced into, etc.) by measurement influencing factors (e.g., error factors, interfering effects, etc.). For example, the capacitance measurements taken by the capacitive sensors may be affected by measurement influencing facts such as changes in sensor geometry (e.g., misalignment of the capacitive sensor, movement in elements of the capacitive sensor, etc.), deposition of material on the capacitive sensor (e.g., buildup on an inside of a conduit, solids deposited on the capacitive sensor from a fluid, etc.), or wear on the capacitive sensor (e.g., a fluid wearing on a surface of the capacitive sensor, a fluid wearing on an inside of a conduit, solids dispersed in a fluid wearing on the capacitive sensor, etc.). Changes in the measurement influencing factors may introduce error into the capacitance measurements taken by the capacitive sensors of the fluid mixture, resulting in errors when determining the fluid permittivity, the composition, and/or the dispersion of the fluid.

As a result, measurement influencing factors such as sensor geometry and material conditions of the capacitive sensors may be controlled carefully during the manufacturing, installation, and calibration processes to ensure the accuracy of the capacitive sensors. However, even if these measurement influencing factors are carefully monitored, changes to the measurement influencing factors may occur later in a life (e.g., an operational life, a length of time that the capacitive sensor is operational, etc.) which may result in a reduced accuracy of the capacitive sensors. In some instances, once the capacitive sensors are installed, there is no ready means for accurately recalibrating the capacitive sensors to account for the changes in the measurement influencing factors. For example, once the capacitive sensors are installed into a pipeline, the capacitive sensors may not be accurately recalibrated without removing the capacitive sensors from the pipeline. Removing the capacitive sensors from the pipeline may be cost prohibitive (e.g., expensive, operationally difficult, etc.), especially for subsea pipelines which may be difficult to access (e.g., require divers to access, require a specialty drillship to access, etc.).

Implementations described herein are related to a capacitive sensor system that does not require a capacitive sensor to be removed from a pipeline to be recalibrated. Instead, the capacitive sensor system described herein (e.g., a dynamic configurable capacitive sensor, a dynamic configurable capacitive sensor array, etc.) uses multiple modes of operation to verify the fluid permittivity measurements taken by the capacitive sensor. As a result of the multiple mode of operation of the capacitive sensor system, an operator can calibrate the capacitive sensor online (e.g., real-time, without removing the capacitive sensor, etc.) to account for the measurement influencing factors affecting the capacitive sensor and ensure that the capacitance measurements taken by the capacitive sensor are within a required accuracy (e.g., above an accuracy threshold, that an error in the capacitance measurements is below an error threshold, etc.). Additionally, the capacitive sensor system described herein is capable of using the capacitive sensor to determine a velocity of a fluid in a conduit. As a result, the operator is able to minimize a variety of sensors required to determine multiple properties of the fluid flowing through the conduit. The capacitive sensor system described herein may allow for an operator to determine a measurement of a relatively small water fraction with high resolution in an oil mixture. Additionally, the capacitive sensor system may allow for the operator to continue to determine the measurement of the relatively small water fraction despite the measurement influencing factors effecting measurements provided by the capacitive sensors of the capacitive sensor system.

Referring generally to the FIGURES, systems, and methods for capacitive sensor calibration (e.g., techniques for calibrating a capacitive sensor, etc.) are shown, according to some embodiments. The capacitive sensor includes electrodes configured to measure capacitances through a fluid flowing through a conduit. In some embodiments, the capacitive sensor is made up of co-planer capacitors (e.g., capacitors with multiple electrodes that share the same plane, etc.).

A control system is configured to calibrate the capacitive sensor based on receiving multiple measurements of capacitances from the capacitive sensor corresponding with the fluid. In some embodiments, each of the measurements of the capacitances corresponds with a different path through the fluid. The control system is configured to determine a fluid property of the fluid based on the measurements of the capacitances. In some embodiments, the fluid property is a permittivity of the fluid. The control system may determine a value of the fluid property that corresponds with each of the measurements of the capacitances from the capacitance sensor and compare the values of the fluid property to determine a variation. In some embodiments, if the variation is above a predetermined threshold, the control system may be configured to receive additional measurements of capacitances of the fluid. In some embodiments, the control system is configured to model the values of the fluid properties to determine a sensor error associated with one of the values of the fluid properties. The sensor error may be utilized by the control system to calibrate the capacitive sensor to reduce the sensor error. In various embodiments, the control system is also configured to utilize the multiple measurements of the capacitances form the capacitive sensor to determine a velocity of the fluid.

### Capacitive Sensor Calibration Techniques

### Fluid Pipeline

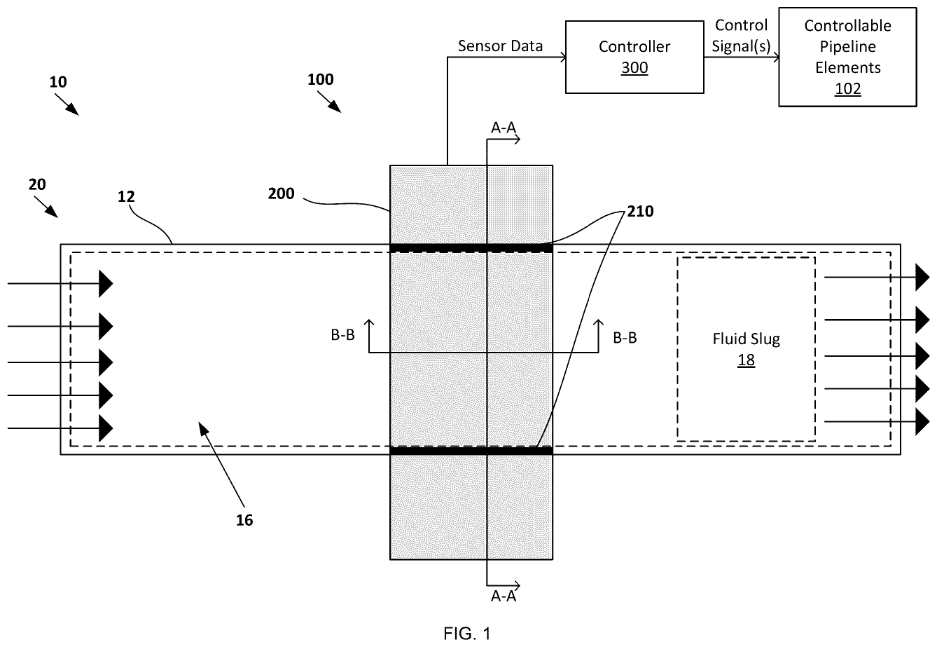

Referring to FIG. 1, a system 10 for monitoring a pipeline 12 (e.g., a pipeline for fluid such as gas or liquid or a mixture of the two, a pipeline for a gas such as a compressible gas, natural gas including methane and contaminants, an acid gas such as carbon dioxide and hydrogen sulfide, or a pipeline for liquids such as natural gas, gasoline, aviation fuel, crude oil, distillates, diesel, butane, propane, ethane, etc.) is shown, according to some embodiments. The system 10 can be configured to monitor one or more conditions of a fluid 16 (e.g., a hydrocarbon, a natural gas, a gas, a liquid/gas mixture, etc.) that flows or travels within the pipeline 12. The system 10 can include a control system 100 that is configured to receive and use sensor inputs from one or more sensing units 200 that measure one or more conditions or properties of the fluid (e.g., temperature, pressure, dynamic pressure, static pressure, flow rate, fluid permissively, etc.) to determine various properties of the fluid (e.g., composition, quality of dispersion, etc.) or to adjust the operation of one or more devices of the system 10 (e.g., to affect the fluid 16 within the pipeline 12). In some embodiments, the pipeline 12 is for a crude oil, natural gas, hydrogen, gasoline, an acid gas (e.g., including a mixture of carbon dioxide and hydrogen sulfide), or other petroleum products including but not limited to mixtures of oil and gas products (e.g., mixtures of hydrocarbons and water, etc.). In other embodiments, the system may be configured to monitor one or more conditions of the fluid 16 in other operations. For example, the system 10 may be configured to monitor one or more conditions of the fluid 16 while the fluid 16 is being stored in a tank, pumped through a pump, or involved in other operations.

The pipeline 12 may be a portion of a pipeline system 20. The pipeline system 20 can be a distribution, manufacturing, or consumption system for distributing the fluid 16, manufacturing the fluid 16, or consuming the fluid 16. In some embodiments, the fluid 16 may change composition throughout different portions of the pipeline system 20 (e.g., a liquid, a liquid/gas mixture, a hydrocarbon, an additive, etc.). In some embodiments, the pipeline system is configured to gather the fluid 16 (e.g., receive gas and/or oil from wells), transmit the fluid 16 (e.g., ship gas and/or oil across the country), and/or distribute the fluid 16 (e.g., distribute gas and/or oil to end customers). In various embodiments, the pipeline system 20 is configured to gather multiple streams of the fluid 16 into a single stream. It should be understood that while FIG. 1 shows only a portion of the pipeline system 20, the pipeline system 20 may be more extensive, and may include any number of pipes, conduits, tubular members, etc. In some embodiments, the control system 100 as shown in FIG. 1 is repeated at various intervals down the pipeline system 20. The fluid 16 (or other petroleum product or mixture) flowing through the pipeline 12 may be modeled as one or more fluid slugs 18 (e.g., one or more fluid parcels, a fluid parcel, a first fluid parcel, a second fluid parcel, etc.). For example, the fluid slug 18 can represent a certain amount, volume, portion, or quantity of the fluid 16 that flows through the pipeline 12.

The control system 100 also includes the sensing unit 200 that includes one or more sensors. In some embodiments, the control system 100 may include any number of sensing units 200 to measure conditions or properties of the fluid 16 at different locations of the pipeline 12 or the pipeline system 20. The sensing unit 200 includes a capacitive sensor 210 configured to measure a capacitance through the fluid 16 that flows through the pipeline 12. In some embodiments, the capacitive sensor 210 is configured to measure multiple of capacitances of the fluid 16 (e.g., a first capacitance, a second capacitance, a third capacitance, etc.) across different paths (e.g., a first path, a second path, a third path, routes, etc.) through the pipeline 12. In some embodiments, the capacitive sensor 210 may be positioned outside of the pipeline 12 and may be configured to measure both the capacitance of the pipeline 12 and the capacitance of the fluid 16. For example, the capacitive sensor 210 may be configured to measure a combined capacitance of the pipeline 12 and the fluid 16 and the control system 100 may be configured to determine the capacitance of the fluid 16 based on the combined capacitance and known properties of the pipeline 12 (e.g., the capacitance of the pipeline 12 may be known, etc.).

In various embodiments, the sensing units 200 may include additional sensors configured to measure additional fluid properties of the fluid 16 that flows through the pipeline 12 (e.g., temperature, velocity or flow rate, pressure, etc.). It should be understood that the sensing unit 200 can include any number of sensors configured to measure other conditions or properties of the fluid 16, or to measure/obtain values of properties or conditions of the fluid 16 that can be used (e.g., by a controller) to estimate or calculate other properties of the fluid 16 (e.g., using a model of a composition of the fluid 16).

It should be understood that the pipeline 12 as described herein may transfer a gas, a liquid, a fluid, etc. In some embodiments, the fluid 16 is a diesel fuel, gasoline, propane, etc. In some embodiments, the fluid 16 is configured to transport different types of gases or substances. For example, the pipeline 12 can be configured to transport both a diesel fuel and gasoline, according to some embodiments. When different gases or liquids or substances are transported through the pipeline 12, the different gases, liquids, or substances may mix at an interface between the different substances (resulting in a slop or transmix mixture).

The control system 100 includes the controller 300 (e.g., a programmable logic controller (PLC), a feedback controller, a processing unit, processing circuitry, etc.) that is configured to obtain sensor data from the sensing unit 200, or from the capacitive sensors 210 of the sensing unit 200. The controller 300 can use the sensor data obtained from the sensing unit 200 to determine one or more properties (e.g., a phase, a composition, a fluid permittivity, etc.) of the fluid 16 that flows within the pipeline 12, can calibrate the sensing unit 200, and can generate control decisions for one or more controllable pipeline elements 102. The controllable pipeline elements 102 may be configured to adjust an operation of the pipeline system 20 (e.g., a shut-off valve or pressure control valve) or to adjust/control one or more properties of the fluid 16 that flows through the pipeline 12 (e.g., adjusting operation of a pump or compressor). In this way, the controller 300 can perform a closed-loop feedback control scheme to adjust operation of the controllable pipeline elements 102 based on real-time or current sensor data obtained from the sensing units 200. In some embodiments, temperature, pressure, flow rate and composition can be controlled by various equipment (e.g., a valve for changing flow composition, heating coil, cooling coil, boiler, heat exchanger, port for inserting or removing material, a compressor or pump for controlling pressure, a mixer for changing homogeneity of the material, etc.).

The controller 300 can also use a model of a composition of the fluid 16 that flows through the pipeline 12 to estimate the composition of the fluid 16 based on the capacitance measurements received from the sensing units 200. The controller 300 can generate the control signal(s) for the controllable pipeline elements 102 to maintain the fluid 16 at a desired composition, a desired phase, or at a desired temperature and pressure. The controller 300 may operate the controllable pipeline elements 102 to maintain the fluid 16 at the desired composition to control a water composition in the fluid 16 (e.g., to minimize a water composition in the fluid 16 to maximize a hydrocarbon composition in the fluid 16, to optimize a water composition in the fluid 16 to promote transportation of the fluid 16 through the pipeline 12, etc.) or to reduce an amount of water within the fluid 16 due to production requirements (e.g., fluid separators unable to handle the fluid 16 with a water composition above a certain level, etc.).

### Controller Diagram

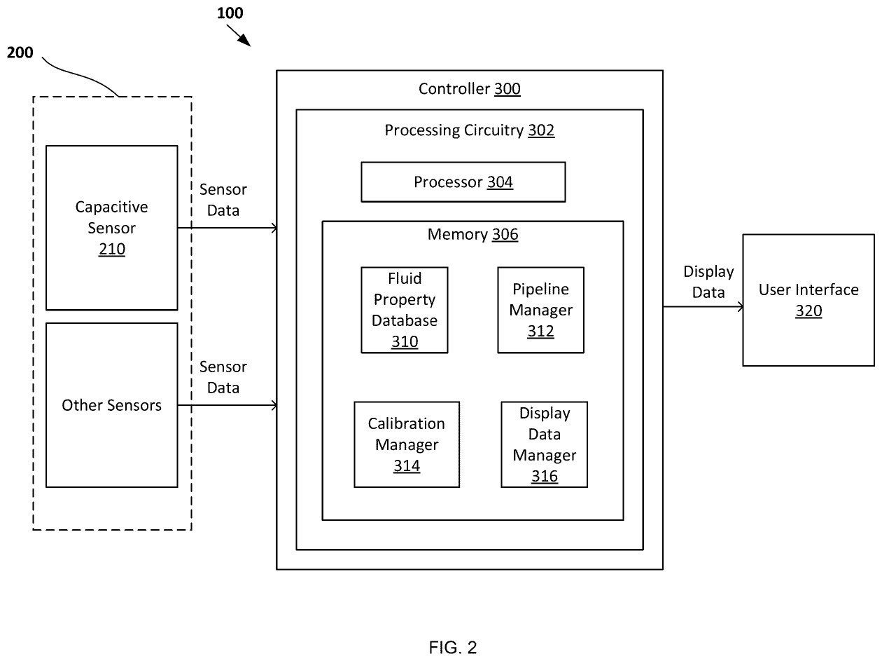

Referring now to FIG. 2, the control system 100 is shown in greater detail, according to some embodiments. The control system 100 includes the sensing units 200, the controller 300 and a user interface 320 (e.g., a device including a display screen, a user input device, etc.). In some embodiments, the controller 300 is configured to obtain sensor inputs from the sensing units 200 including the capacitance through the fluid 16 from the capacitive sensor 210. In various embodiments, the controller 300 is also configured to obtain additional sensor inputs from the sensing units 200 including the temperature of the fluid 16, the pressure of the fluid 16, the flow rate (e.g., velocity, volumetric flow rate, etc.), and/or the composition of the fluid 16. In various embodiments, the controller 300 is configured to obtain sensor inputs from multiple of the sensing units 200.

In some embodiments, the controller 300 can use the sensor inputs in a model to determine a calibration of the sensing unit 200. In some embodiments, the controller 300 can use the sensor inputs in a model to determine a velocity of the fluid 16, a composition of the fluid 16, and/or control operations or control signals for the controllable pipeline elements 102 to maintain the fluid 16 within or at a desired composition, to maintain the fluid 16 at a desired velocity etc. The controller 300 can also generate and output display information for the user interface 320 (e.g., an X-Y plot, a table, etc.) so that the user interface 320 can operate to display current conditions of the fluid 16 in the pipeline 12 for an operator or a technician.

The controller 300 includes processing circuitry 302 including a processor 304 and memory 306. The processor 304 can be a general purpose or specific purpose processor, an application specific integrated circuit (ASIC), one or more field programmable gate arrays (FPGAs), a group of processing components, or other suitable processing components. The processor 304 may be configured to execute computer code and/or instructions stored in the memory 306 or received from other computer readable media (e.g., CDROM, network storage, a remote server, etc.).

The memory 306 can include one or more devices (e.g., memory units, memory devices, storage devices, etc.) for storing data and/or computer code for completing and/or facilitating the various processes described in the present disclosure. The memory 306 can include random access memory (RAM), read-only memory (ROM), hard drive storage, temporary storage, non-volatile memory, flash memory, optical memory, or any other suitable memory for storing software objects and/or computer instructions. The memory 306 can include database components, object code components, script components, or any other type of information structure for supporting the various activities and information structures described in the present disclosure. The memory 306 can be communicably connected to the processor 304 via the processing circuitry 302 and can include computer code for executing (e.g., by the processor 304) one or more processes described herein.

The memory 306 is shown to include a database 310, a pipeline manager 312, a calibration manager 314, and a calibration manager 314. The database 310 can store fluid parameters and/or fluid models that can be used by the pipeline manager 312 to determine fluid properties of the fluid 16 based on the sensor data received from the sensing unit 200. For example, the database 310 can store models relating capacitance and fluid permittivity of different fluids (e.g., water, oil, gases, etc.) that can be used by the pipeline manager 312 to derive the fluid permittivity of the fluid 16 based on the capacitance measurement of the capacitive sensor 210. As another example, the database 310 can store models of fluid mixtures relating the composition of the fluid mixtures to fluid permittivity that can be used by the pipeline manager 312 to determine the composition of the fluid 16 based on the capacitance measurement of the capacitive sensor 210.

In some instances, the pipeline manager 312 may utilize the models of the fluid mixtures relating the composition of the fluid mixtures to fluid permittivity to determine water content in fluid mixtures. For example, the pipeline manager 312 may utilize the models of the fluid mixtures relating the composition of the fluid mixtures to fluid permittivity to determine a water content in the fluid 16. In some embodiments, the pipeline manager 312 may be configured to determine a relatively small water fraction (e.g., less than 25% water, less than 15% water, less than 5% water, less than 1% water, etc. of an oil mixture (e.g., a mixture containing oil and water, a mixture containing oil and various other fluids, a fluid mixture, etc.) based on the sensor data received from the sensing unit 200. For example, the database 310 may include a permittivity of oil and a permittivity of water, and the pipeline manager 312 may utilize the sensor data received from the sensing unit 200 including a capacitance measurement through a mixture of oil and water (e.g., a mixture containing oil and water, etc.), the permittivity of oil, and the permittivity of water to determine a fraction of water in the mixture of water or oil. As another example, the database 310 may include various permittivities of fluids and the pipeline manager 312 may utilize the sensor data received from the sensing unit 200 including a capacitance measurement through a fluid mixture containing a selection of fluids corresponding to the various permittivities of fluids and the various permittivities of the fluids included in the fluid mixture to determine a fraction of one of the fluids in the fluid mixture. In some embodiments, the database 310 includes a series of lookup tables that can be used by the pipeline manager 312 to determine fluid properties of the fluid 16.

In some embodiments, the database 310 includes geometry of the capacitive sensor 210. For example, the database 310 may include positional relationships of components of the capacitive sensor 210, thicknesses of components of the capacitive sensor 210, material data relating to materials making up components of the capacitive sensor 210, or other information corresponding to the capacitive sensor 210. The pipeline manager 312 and/or the calibration manager 314 may utilize the information corresponding to the capacitive sensor 210 to determine fluid properties of the fluid 16 and/or perform calibration operations associated with the capacitive sensor 210.

In some embodiments, the pipeline manager 312 is configured to use any of the fluid properties of the fluid 16 as determined based on the sensor data from the sensing unit 200 to determine control operations for the controllable pipeline elements 102 in order to control the flow of the fluid 16 through the pipeline 12. For example, the pipeline manager 312 may determine control operations to maintain the flow of the fluid 16 through the pipeline 12 (e.g., the controllable pipeline elements 102 include a valve and the control operation is to open or close the valve, etc.), change a composition of the fluid 16 in the pipeline 12 (e.g., the controllable pipeline elements 102 include a mixer and the control operation is to change an operation of the mixer, etc.), etc. and provide control signals corresponding to the control operations to the controllable pipeline elements 102. In various embodiments, the pipeline manager 312 is also configured to determine control operations by providing control signals to multiple of the controllable pipeline elements 102 of the pipeline 12.

The calibration manager 314 is configured to receive the sensor data from the sensing unit 200 and determine if variations in the sensor data are above predetermined thresholds (e.g., a predetermined variation threshold, a predetermined error threshold, etc.). For example, the sensing unit 200 may be configured to measure a first temperature at a first temperature sensor and a second temperature at a second temperature sensor. The calibration manager 314 may receive the sensor data from the sensing unit 200 corresponding to the first temperature and the second temperature and compare the first temperature and the second temperature to determine a variation between the first temperature and the second temperature. The calibration manager 314 may compare the variation to a predetermined threshold to determine if the sensor data contains errors.

According to some embodiments, the calibration manager 314 is configured to determine if variations in the sensor data received from the capacitive sensor 210 are above predetermined thresholds. For example, the pipeline manager 312 may receive a first capacitance measured along a first path through the fluid 16 and a second capacitance measured along a second path through the fluid 16 from the capacitive sensor 210 of the sensing unit 200. The pipeline manager 312 may determine a first fluid permittivity (e.g., a first value of a fluid property, etc.) corresponding to the first capacitance and a second fluid permittivity (e.g., a second value of the fluid property, etc.) corresponding to the second capacitance and provide the first fluid permittivity and the second fluid permittivity to the calibration manager 314. The calibration manager 314 may compare the first fluid permittivity and the second fluid permittivity to determine a variation between the first fluid permittivity and the second fluid permittivity. The calibration manager 314 may compare the variation to a predetermined threshold to determine if the capacitive sensor 210 has been affected by a measurement influencing factor (e.g., the orientation of the capacitive sensor 210 has changed, material buildup has occurred at the capacitive sensor 210, erosion has occurred at the capacitive sensor 210, etc.), resulting in an error in either the first capacitance or the second capacitance.

The calibration manager 314 is configured to determine a sensor error associated with a sensor of the sensing unit 200, according to some embodiments. The sensor error may correspond with an error in a measurement of one of the sensors of the sensing unit 200. For example, the calibration manager 314 may determine that a temperature sensor has a sensor error of twelve degrees after determining that the temperature measurement of the temperature sensor has an error of twelve degrees. The calibration manager 314 may utilize the sensor data from the sensing unit 200 and/or the data of the database 310 to determine the sensor error. For example, the sensing unit 200 may be configured to measure a first temperature at a first temperature sensor, a second temperature at a second temperature sensor, and a third temperature at a third temperature sensor. The calibration manager 314 may receive the sensor data from the sensing unit 200 corresponding to the first temperature, the second temperature, and the third temperature and model the first temperature, the second temperature, and the third temperature to determine a sensor error associated with at least one of the first temperature, the second temperature, or the third temperature. The sensor error may indicate a calibration issue with the temperature sensor that recorded the temperature associated with the sensor error. For example, if the sensor error is associated with the first temperature, the sensor error may indicate a calibration issue with the first sensor. In some embodiments, the calibration manager 314 may compare the sensor error to a predetermined error threshold to determine if the sensor error is significant (e.g., if the sensor error will have a noticeable effect on the sensor error, if the sensor error will affect operations, etc.).

According to some embodiments, the calibration manager 314 is configured to determine a sensor error associated with the capacitive sensor 210. For example, the pipeline manager 312 may receive a first capacitance measured along a first path through the fluid 16, a second capacitance measured along a second path through the fluid 16, and a third capacitance measured along a third path through the fluid 16 from the capacitive sensor 210 of the sensing unit 200. The pipeline manager 312 may determine a first fluid permittivity (e.g., a first value of a fluid property, etc.) corresponding to the first capacitance, a second fluid permittivity (e.g., a second value of the fluid property, etc.) corresponding to the second capacitance, and a third fluid permittivity (e.g., a third value of the fluid property, etc.) corresponding to the third capacitance and provide the first fluid permittivity, the second fluid permittivity, and the third fluid permittivity to the calibration manager 314. The calibration manager 314 may model the first fluid permittivity, the second fluid permittivity, and the third fluid permittivity to determine a sensor error associated with at least one of the first fluid permittivity, the second fluid permittivity, or the third fluid permittivity. The sensor error may indicate a calibration issue with the capacitive sensor 210.

According to some embodiments, the calibration manager 314 is configured to determine the measurement influencing factor associated with the sensor error of the sensor of the sensing unit 200. For example, if the sensor error is associated with a first temperature sensor and a first temperature corresponding to the first temperature sensor is below a second temperature corresponding to a second temperature sensor and a third temperature corresponding to a third temperature sensor, the calibration manager 314 may determine that the measurement influencing factor associated with the sensor error is an age of the first temperature sensor based on test data of previous temperature sensors indicating that temperatures read by temperature sensors decrease as the temperature sensor ages.

According to some embodiments, the calibration manager 314 is configured to determine the measurement influencing factor associated with the sensor error of the capacitive sensor 210. The calibration manager 314 may determine the measurement influencing factor based on relationships between fluid permittivities corresponding to capacitances measured by the capacitive sensor 210. For example, the calibration manager 314 may determine that the measurement influencing factor associated with the sensor error is deposition of a material on the capacitive sensor 210 based on the fluid permittivity associated with the sensor error being higher or lower than other fluid permittivity's not associated with the sensor error. In some embodiments, the calibration manager 314 may additionally determine the measurement influencing factor associated with the sensor error based on a configuration of the calibration manager 314. For example, if the sensor error is associated with a cross stream capacitance measurement and a first fluid permittivity associated with the sensor error is lower than a second fluid permittivity associated with a downstream capacitance measurement, the calibration manager 314 may determine that the measurement influencing factor is erosion based on the first fluid permittivity associated with the cross stream capacitance decreasing relative to the second fluid permittivity associated with the downstream capacitance measurement.

In some embodiments, the calibration manager 314 is configured to determine a location of the measurement influencing factor associated with the sensor error. For example, the calibration manager 314 may determine the location of a deposit of a material on the capacitive sensor 210 based on the relationships between the fluid permittivities corresponding to the capacitances measured by the capacitive sensor 210. In some embodiments, the calibration manager 314 may determine the material deposited on the capacitive sensor 210. For example, the calibration manager 314 may determine a permittivity of the material using the capacitances measured by the capacitive sensor 210 and may utilize the database 310 to determine the material that corresponds with the permittivity. Determining the material deposited on the capacitive sensor 210 may allow for an operator to perform a mitigation technique that targets removal of the material. For example, if tar is deposited on the capacitive sensor 210, the operator may inject a solvent into the fluid 16 that is configured to dissolve the tar such that the deposit is removed from the capacitive sensor 210. As another example, the calibration manager 314 may determine the location of erosion on the capacitive sensor 210 based on the relationships between the fluid permittivities corresponding to the capacitances measured by the capacitive sensor 210. Determining the location of the erosion may allow for the calibration manager 314 to calibrate the capacitive sensor 210 to mitigate the effects of the erosion on the capacitances measured by the capacitive sensor 210. Additionally, the location of the erosion may allow for the operator to make operational decisions, such as replacing a section of the pipeline 12 that contains the erosion or changing operational conditions to reduce further erosion in the capacitive sensor 210.

The calibration manager 314 is further configured to calibrate the sensor of the sensing unit 200 associated with the sensor error to reduce the sensor error, according to some embodiments. For example, the calibration manager 314 may calibrate the sensor relative to readings of other sensors not associated with the sensor error to reduce the sensor error. In some embodiments, the calibration manager 314 may create a filter for the sensor data from the sensor associated with the sensor error to reduce the sensor error. For example, if a sensor error associated with a temperature sensor is twelve degrees below an actual temperature, the calibration manager 314 may create a filter for the sensor data from the temperature sensor to increase a temperature received from the temperature sensor by twelve degrees before processing the sensor data.

In some embodiments, the calibration manager 314 is further configured to calibrate the capacitive sensor 210 of the sensing unit 200 to reduce the sensor error when the sensor error is associated with the capacitive sensor 210. For example, the calibration manager 314 may calibrate a first capacitance measurement of the capacitive sensor 210 associated with the sensor error relative to a second capacitance measurement or a third capacitance measurement that are not associated with the sensor error. In some embodiments, the calibration manager 314 may create a filter for the sensor data from the capacitive sensor 210 corresponding to a capacitive measurement associated with the sensor error. For example, if a sensor error is associated with a capacitive measurement of the capacitive sensor 210, the filter may modify the first capacitive measurement to reduce the sensor error before the calibration manager 314 processes the capacitive measurement.

Referring still to FIG. 2, the display data manager 316 is configured to generate display data and provide the display data to the user interface 320, according to some embodiments. The user interface 320 may be a remote device, a user device, a display screen, etc., according to some embodiments. In some embodiments, the display data generated by the display data manager 316 includes elements corresponding to variations between measurements of the sensors of the sensing unit 200. The display data can also include any of the sensor data obtained by the sensing unit 200 or the sensor errors of the sensors of the sensing unit 200, according to some embodiments. In various embodiments, the display data includes calibration actions that may be performed by the calibration manager 314 to reduce the sensor error of sensors of the sensing unit 200. For example, the display data may include that calibrating a sensor may reduce the sensor error associated with the sensor. As another example, the display data may include that replacing a sensor will reduce the sensor error associated with the sensor. In some embodiments, the display data also includes the control decisions made by the pipeline manager 312.

### Capacitance Sensor Implementation

In some embodiments, a capacitive sensor includes a first electrode, a second electrode spaced from the first electrode, and an electronic circuit electrically coupled to the first electrode and the second electrode. The electronic circuit is configured to measure a capacitance between the first electrode and the second electrode. The capacitance between the first electrode and the second electrode is a function of a geometry of the first electrode, a geometry of the second electrode, a configuration of the first electrode relative to the second electrode, and a material between the first electrode and the second electrode (e.g., a material surrounding the first electrode and the second electrode, etc.). The capacitance is defined by an amount of charge that can be stored between the first electrode and the second electrode per a difference in an electric potential in the first electrode and the second electrode, or by the charge related to the mutual flux between the first electrode and the second electrode and the voltage between the first electrode and the second electrode. Typically, instead of the charge, the complex impedance between electrode voltage and current is measured, either with an excitation with an AC sine wave or in a switched capacitor topology. In other embodiments, the excitation configured as a voltage differential between two electrodes. To ensure only charge related to mutual flux is measured the measurement electrode must be isolated against any surrounding material that is on ground potential.

For example, a parallel plate capacitor has two plates that are separated by a given distance. A material is positioned between the two plates. The capacitance of the parallel plate capacitor may be calculated using the following formula:

$$C = \frac{ε \times A}{d}$$

where C is the capacitance, ε is the permittivity of the material between the two plates, A is an overlapping surface area of the two plates, and d is the distance between the two plates. If the overlapping surface area of the two plates and distance between the two plates are known and the capacitance of the parallel plate capacitor is measured using a capacitive sensor, the permittivity of the material between the two plates may be determined. The design of the parallel plate capacitor may be a basis or reference case for a capacitance sensor as the parallel plate capacitor results in a largely volumetric average measurement of the material dielectric properties.

The capacitive sensor may use alternative electrode geometries to determine permittivity of material proximate the capacitive sensor. For example, the capacitive sensor may include a first electrode and a second electrode that are co-planer. The first electrode and the second electrode may be thin and be positioned side by side with each other. In such a co-planer design, the dielectric properties (e.g., the permittivity, etc.) of materials positioned between the first electrode and the second electrode are the main factor in the measured capacitance. For coplanar electrodes, the electrical field lines are concentrated near the gap between the first electrode and the second electrode, making this gap the area of highest sensitivity. However, the electric properties of the materials adjacent the first electrode and the second electrode also factor into the measured capacitance.

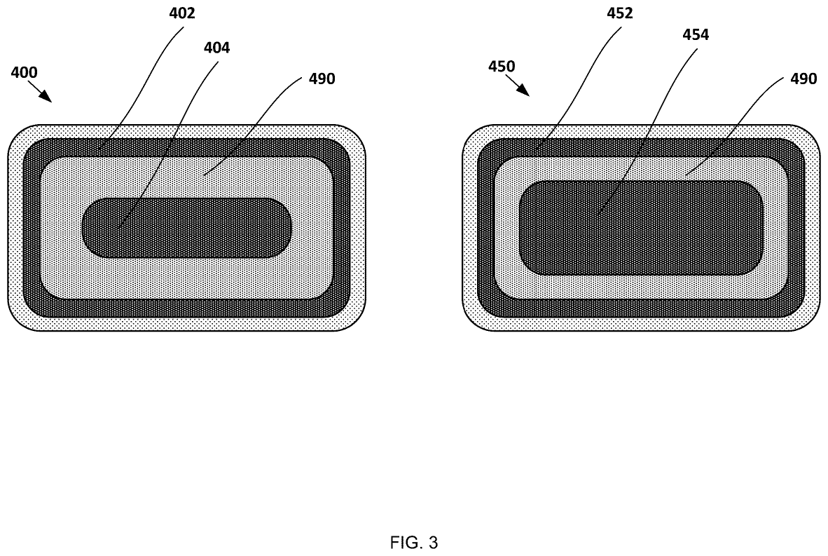

Referring now to FIG. 3, an embodiment of a first capacitive sensor 400 and a second capacitive sensor 450 demonstrate how a first capacitance measured by the first capacitive sensor 400 across a fluid 490 may be compared with a second capacitance measured by the second capacitive sensor 450 across the fluid 490 to detect uncertainty and error of the first capacitive sensor 400 or the second capacitive sensor 450. The first capacitive sensor 400 includes a first electrode 402 and a second electrode 404. The first electrode 402 defines an opening through the first electrode 402 configured to receive the fluid 490. The second electrode 404 is positioned in the opening defined by the first electrode 402 such that a first distance between an outer surface of the second electrode 404 and an inner surface of the first electrode 402 is constant. For example, the first distance may be 7 mm. The first capacitive sensor 400 may be configured to measure a first capacitance between the first electrode 402 and the second electrode 404, which may then be used to determine a first permittivity of the fluid 490. An equation for the first capacitance may be expressed as:

$${C}_{meas \_ 1} = {C}_{sensor \_ 1} + {k}_{1} {ε}_{f \_ 1}$$

where C_{meas_1} is the first capacitance measured by the first capacitive sensor 400, C_{sensor_1} is a capacitance associated with the field lines that pass through the first electrode 402 and the second electrode 404, ε_{f_1} is the first permittivity of the fluid 490, and k₁ is a geometry factor for the first capacitive sensor 400.

The second capacitive sensor 450 includes a first electrode 452 and a second electrode 454. The first electrode 452 defines an opening through the first electrode 452 configured to receive the fluid 490. The second electrode 454 is positioned in the opening defined by the first electrode 452 such that a second distance between an outer surface of the second electrode 454 and an inner surface of the first electrode 452 is constant. For example, the first distance may be 4 mm. The second capacitive sensor 450 may be configured to measure a second capacitance between the first electrode 452 and the second electrode 454, which may then be used to determine a second permittivity of the fluid 490. An equation for the second capacitance may be expressed as:

$${C}_{meas \_ 2} = {C}_{sensor \_ 2} + {k}_{2} {ε}_{f \_ 2}$$

where C_{meas_2} is the second capacitance measured by the second capacitive sensor 450, C_{sensor_2} is a capacitance associated with the field lines that pass through the first electrode 452 and the second electrode 454, ε_{f_2} is the second permittivity of the fluid 490, and k₂ is a geometry factor for the second capacitive sensor 450.

Referring to both of the equations above, it can be understood that if C_{sensor_1}, C_{sensor_2}, k₁ and k₂ are known (e.g., by means of calibration, by calculation, etc.), C_{meas_1} is measured by the first capacitive sensor 400, and C_{meas_2} is measured by the second capacitive sensor 450, then both equations can be solved for ε_{f} to determine the first permittivity and the second permittivity of the fluid 490. When the values of ε_{f_1} and ε_{f_2} obtained from C_{meas_1} and C_{meas_2} respectively agree with one another within a given tolerance or uncertainty (e.g., a variation between ε_{f_1} and ε_{f_2} is below a variation threshold, etc.), then the measurement results from the first capacitive sensor 400 and the second capacitive sensor 450 may be considered verified. When the values of ε_{f_1} and ε_{f_2} obtained from C_{meas_1} and C_{meas_2} respectively do not agree with each other outside of the given tolerance or uncertainty (e.g., the variation between ε_{f_1} and ε_{f_2} is above the variation threshold, etc.), then the measurement results from the first capacitive sensor 400 and the second capacitive sensor 450 may be considered unverified (e.g., the measurement result from the first capacitive sensor 400 or the second capacitive sensor 450 is abnormal and should be corrected, etc.). In some embodiments, the capacitive sensor 210 may be configured as a combination of the first capacitive sensor 400 and the second capacitive sensor 450.

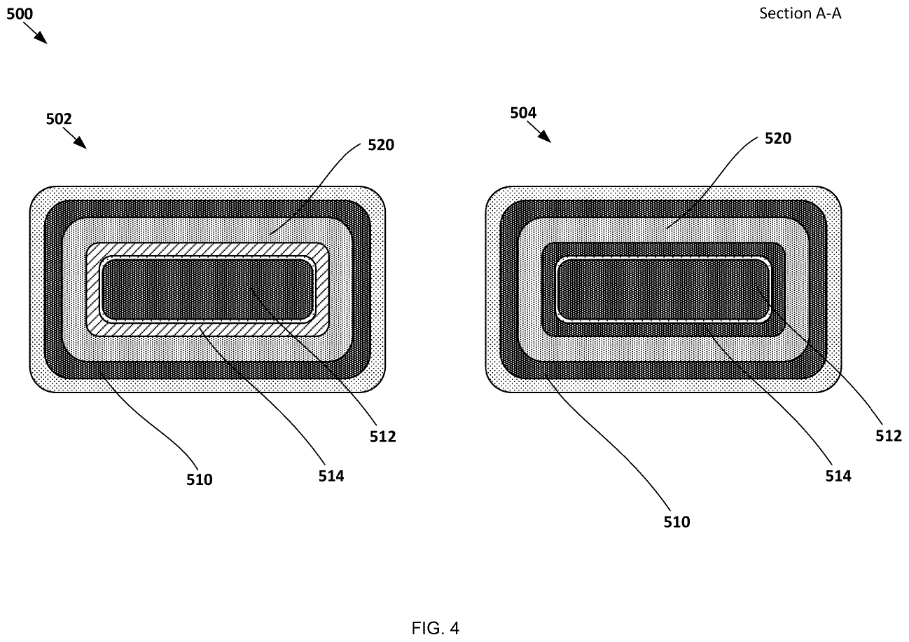

Referring to FIG. 4, an example embodiment of a capacitive sensor 500 is shown in a first operational mode 502 and a second operational mode 504 that allow for the capacitive sensor 500 to function similarly to both the first capacitive sensor 400 and the second capacitive sensor 450. For example, the capacitive sensor 500 may be configured to measure a first capacitance along a first path while in the first operational mode 502 and to measure a second capacitance along a second path while in the second operational mode 504. In some embodiments, the capacitive sensor 210 may be configured as the capacitive sensor 500. The capacitive sensor 500 includes an outer electrode 510 (e.g., a first electrode, etc.), a first inner electrode 512 (e.g., a second electrode, etc.), and a second inner electrode 514 (e.g., a third electrode, etc.). The outer electrode 510 defines an opening through the outer electrode 510 configured to receive a fluid 520. In some embodiments, the fluid 520 may flow through opening defined by the outer electrode 510. The first inner electrode 512 is positioned in the opening defined by the outer electrode 510 such that a first distance between an outer surface of the first inner electrode 512 and an inner surface of the outer electrode 510 is constant. For example, the first distance may be 7 mm. The second inner electrode 514 is also positioned in the opening defined by the outer electrode 510 around the first inner electrode 512 such that a second distance between an outer surface of the first inner electrode and the inner surface of the outer electrode 510 is constant and is less than the first distance. The capacitive sensor 500 is configured to supply an excitation (e.g., a voltage differential, an alternating voltage differential, etc.) to the outer electrode 510 and the first inner electrode 512 while in the first operational mode 502 to measure a first capacitance across the fluid 520 and the second inner electrode 514 positioned between the first inner electrode 512 and the outer electrode 510, which may be used to determine a first permittivity of the fluid 520. The capacitive sensor 500 is configured to supply the excitation to the outer electrode 510, the first inner electrode 512, and the second inner electrode 514 while in the second operational mode 504 to measure a second capacitance through the fluid 520 positioned between the second inner electrode 514 and the outer electrode 510, which may be used to determine a second permittivity of the fluid 520.

The capacitive sensor 500 may be configured to alternate between the first operational mode 502 and the second operational mode 504 in order to determine the first permittivity of the fluid 520 and the second permittivity of the fluid 520 using equations similar to the equations discussed above relating to the first capacitive sensor 400 and the second capacitive sensor 450. The first permittivity and the second permittivity may also be compared to determine a variation between the first permittivity and the second permittivity. If the variation is above a variation threshold, then the results from the capacitive sensor 500 may be considered unverified. When the results from the capacitive sensor 500 are considered unverified, it may be an indication that the capacitive sensor 500 should be calibrated.

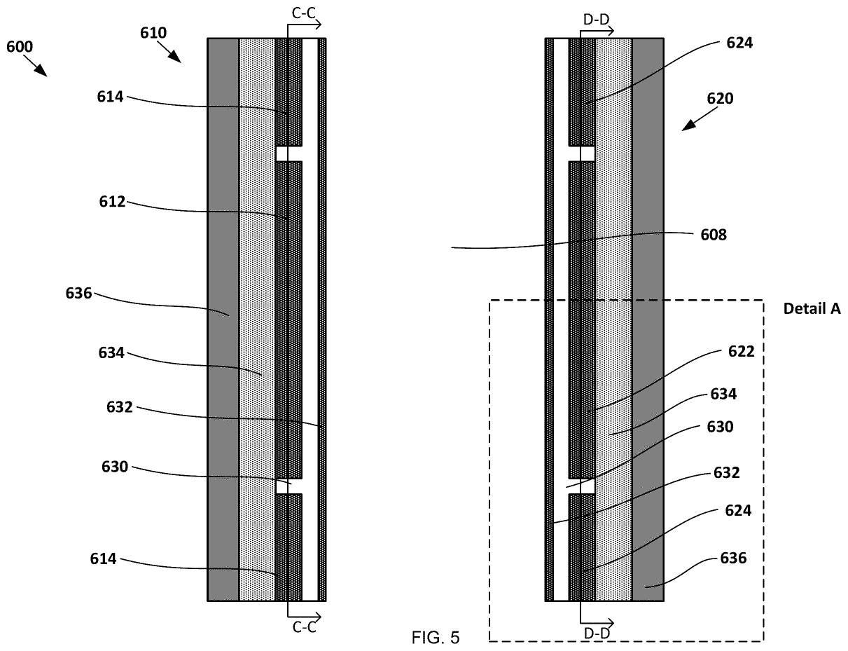

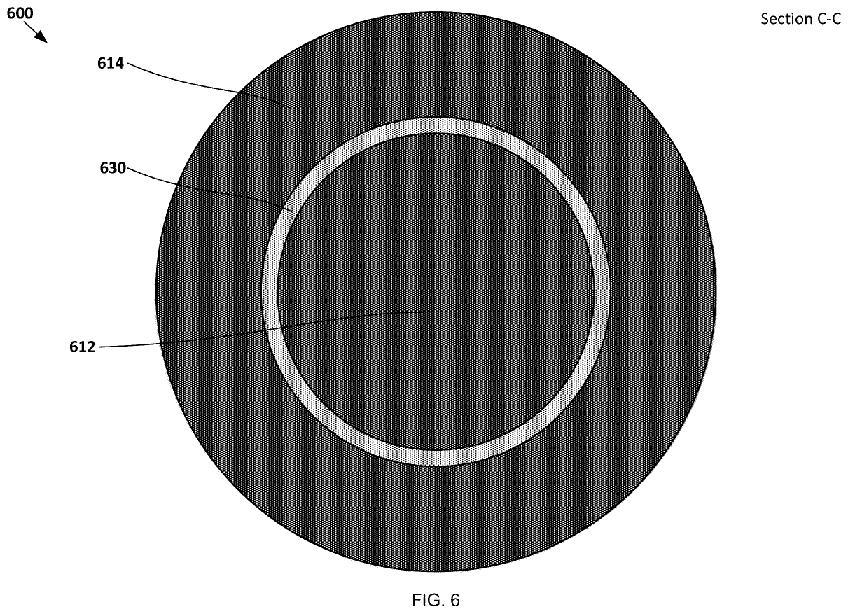

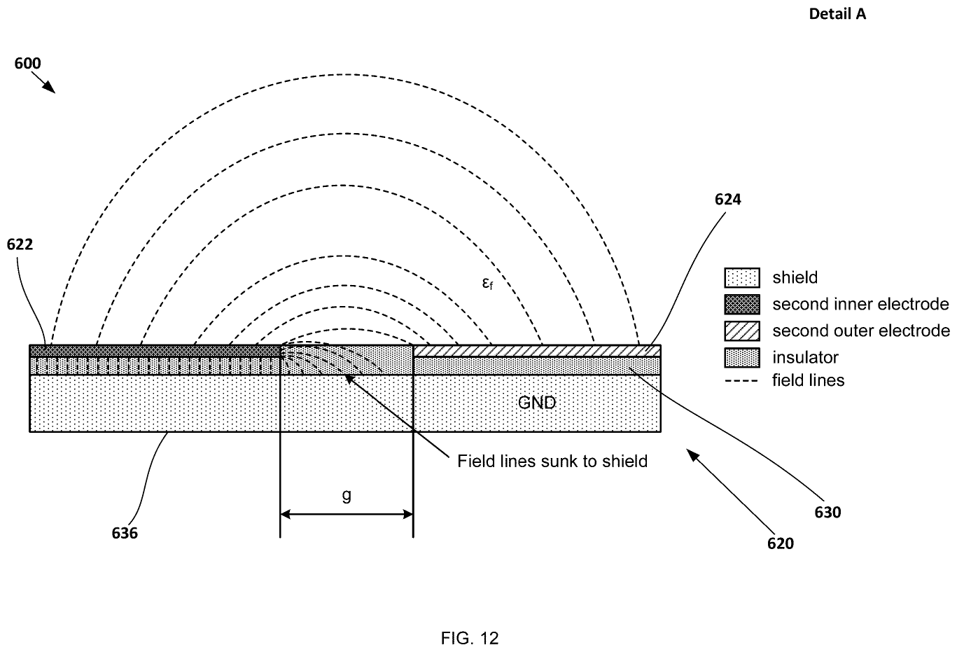

Referring to FIGS. 5-13, an example embodiment of a capacitive sensor 600 is shown. In some embodiments, the capacitive sensor 210 may be configured as the capacitive sensor 600. The capacitive sensor 600 may be operated in a first operational mode 602, a second operational mode 604, and a third operational mode 606 and is configured to measure a capacitance associated with a fluid 608. The fluid 608 may be flowing through the capacitive sensor 600 and/or may be the fluid 16 flowing through the pipeline 12. When the capacitive sensor 600 is in the first operational mode 602, the capacitive sensor 600 may be configured to measure a first capacitance associated with the fluid 608 along a first path. When the capacitive sensor 600 is in the second operational mode 604, the capacitive sensor 600 may be configured to measure a second capacitance associated with the fluid 608 along a second path. When the capacitive sensor 600 is in the third operational mode 606, the capacitive sensor 600 may be configured to measure a third capacitance associated with the fluid 608 along a third path. In some embodiments, the first operational mode 602 is considered a primary operational mode (e.g., a measurement mode, etc.) and the second operational mode 604 and the third operational mode 606 are considered secondary operational modes (e.g., a verification mode, a calibration mode, etc.). The first operational mode 602, the second operational mode 604, and the third operational mode 606 may allow for the capacitive sensor 600 to compare different measured permittivities of the fluid 608 in order to verify the measurements of the capacitive sensor 600. Additionally, the first operational mode 602, the second operational mode 604, and the third operational mode 606 may allow for the capacitive sensor 600 to continue to measure capacitances associated with the fluid 16 when one of the first operational mode 602, the second operational mode 604, and the third operational mode 606 are determined to be inaccurate.

The capacitive sensor 600 includes a first electrode assembly 610 and a second electrode assembly 620 separated by a fluid-filled gap filled by the fluid 608. In some embodiments, the capacitive sensor 600 may be configured to be installed as part of the pipeline 12. For example, the first electrode assembly 610 may be configured to be installed on a first side of the pipeline 12 and the second electrode assembly 620 may be configured to be installed on a second side of the pipeline 12 radially opposing the first electrode assembly 610 such that the first electrode assembly 610 and the second electrode assembly 620 are positioned on opposite sides of the fluid 16 flowing through the pipeline 12.

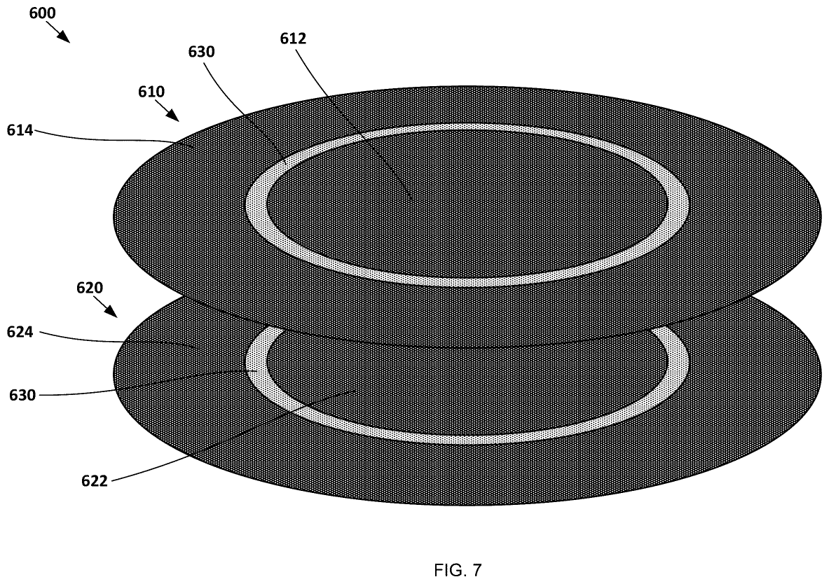

The first electrode assembly 610 is configured as a first co-planar capacitor that includes a first inner electrode 612 and a first outer electrode 614, according to some embodiments. According to the example embodiment shown in FIG. 7, the first inner electrode 612 is a circular electrode and defines a first electrode plane C-C through the first inner electrode 612. The first outer electrode 614 is ring electrode that is configured to surround the first inner electrode 612 along the first electrode plane C-C. In other embodiments, the first inner electrode 612 and the first outer electrode 614 are electrodes with other shapes (e.g., ovular, rectangular, triangular, etc.). In still other embodiments, the first outer electrode 614 may not surround the first inner electrode 612. For example, the first outer electrode 614 may be positioned proximate the first inner electrode 612 along the first electrode plane C-C.

The second electrode assembly 620 is configured as a second co-planar capacitor that includes a second inner electrode 622 and a second outer electrode 624, according to some embodiments. In other embodiments, the second electrode assembly 620 is configured as a single capacitor that includes the second inner electrode 622 and no additional electrodes. According to the example embodiment shown in FIG. 7, the second inner electrode 622 is a circular electrode and defines a second electrode plane D-D through the second inner electrode 622. The second outer electrode 624 is ring electrode that is configured to surround the second inner electrode 622 along the second electrode plane D-D. In other embodiments, the second inner electrode 622 and the second outer electrode 624 are electrodes with other shapes (e.g., ovular, rectangular, triangular, etc.). In still other embodiments, the second outer electrode 624 may not surround the second inner electrode 622. For example, the second outer electrode 624 may be positioned proximate the second inner electrode 622 along the second electrode plane D-D.

Referring to FIG. 5, the first electrode assembly 610 and the second electrode assembly 620 also include an insulator 630, according to some embodiments. For the first electrode assembly 610, the insulator 630 is positioned between the first inner electrode 612 and the first outer electrode 614 and is configured to electrically insulate the first inner electrode 612 and the first outer electrode 614. For the second electrode assembly 620, the insulator 630 is positioned between the second inner electrode 622 and the second outer electrode 624 and is configured to electrically insulate the second inner electrode 622 and the second outer electrode 624. In some embodiments, the insulator 630 is also positioned between (i) the first inner electrode 612, the first outer electrode 614, the second inner electrode 622, and the second outer electrode 624 and (ii) the fluid 608 to electrically isolate the fluid 608 from the electrodes.

In some embodiments, the first electrode assembly 610 and the second electrode assembly 620 also include an inner sheath 632 positioned along an inside surface of the first electrode assembly 610 and the second electrode assembly 620 configured to contact the fluid 608. The inner sheath 632 may be configured to protect the first electrode assembly 610 and the second electrode assembly 620 from the fluid 608. For example, the fluid 608 may be corrosive and the inner sheath 632 may be formed of a corrosion resistant material. As another example, the fluid 608 may include solid particles that may result in an increased rate of erosion of material that comes into contact with the fluid 608 and the inner sheath 632 may be formed of an erosion resistant material configured to reduce the rate of erosion of the first electrode assembly 610 and the second electrode assembly caused by the fluid 608.

In some embodiments, the first electrode assembly 610 and the second electrode assembly 620 may also include a control layer 634. The control layer 634 may be configured to selectively provide electrical excitement (e.g., a voltage, an alternating voltage, a sinusoidal voltage, etc.) to the first inner electrode 612, the first outer electrode 614, the second inner electrode 622, and the second outer electrode 624 in order to create a differential between each of the electrodes, resulting in electrical fields forming between each of the electrodes. For example, the control layer 634 may include wiring electrically coupled between each of the first inner electrode 612, the first outer electrode 614, the second inner electrode 622, and the second outer electrode 624 and the controller 300 configured to selectively provide electrical power to each of the electrodes. Additionally, the control layer 634 may be communicatively coupled between each of the first inner electrode 612, the first outer electrode 614, the second inner electrode 622, and the second outer electrode 624 and the controller 300 and may be configured to communicate measurements of capacitance from the first inner electrode 612, the first outer electrode 614, the second inner electrode 622, and the second outer electrode 624 to the controller 300. In some embodiments, the controller 300 may provide control signals through the control layer 634 to alternate the capacitive sensor 600 between the first operational mode 602, the second operational mode 604, and the third operational mode 606.

In some embodiments, the first electrode assembly 610 and the second electrode assembly 620 may also include a shield 636. The shield 636 is configured to block electrical fields through the insulator 630 produced between the first inner electrode 612, the first outer electrode 614, the second inner electrode 622, and the second outer electrode 624. In some embodiments, the shield 636 may be configured as a ground (e.g., provide a ground to the excitement provided by the control layer 634, etc.). In some embodiments, the shield 636 is configured to protect an outside surface of the first electrode assembly 610 and the second electrode assembly 620 that is opposite of the inside surface of the first electrode assembly 610 and the second electrode assembly 620. The shield 636 may be configured to protect the first electrode assembly 610 and the second electrode assembly 620 from objects coming in contact with the first electrode assembly 610 and the second electrode assembly 620 and damaging the first electrode assembly 610 and the second electrode assembly 620. For example, the shield 636 may be a layer of metal configured to resist damage. In other embodiments, the shield 636 may be formed of a rubber or a plastic. In some embodiments, the shield 636 may be continuous. In embodiments, the shield 636 may be formed of several separate elements.

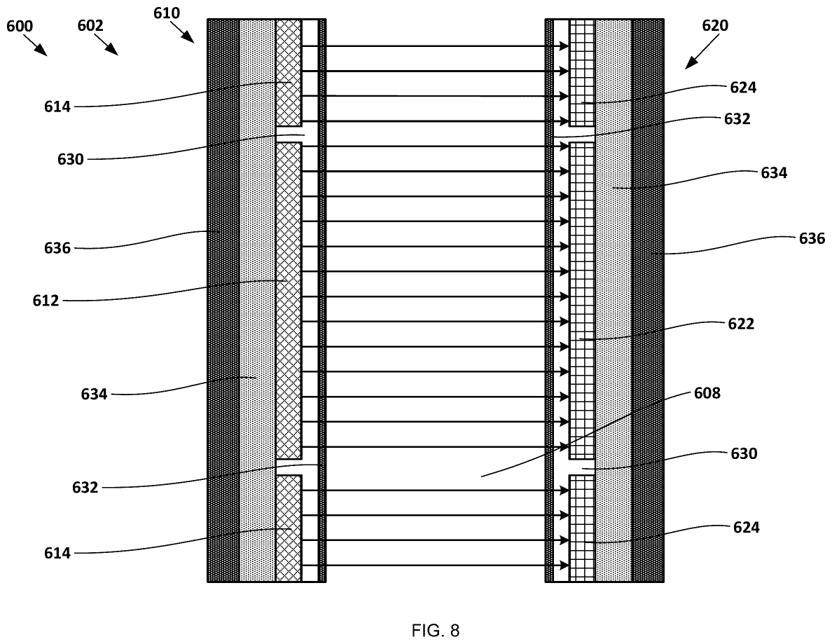

Referring now to FIG. 8, the first operational mode 602 of the capacitive sensor 600 is shown. In the first operational mode 602, the first inner electrode 612 and the first outer electrode 614 are supplied with an excitement (e.g., through the control layer 634, via the controller 300, etc.) such that an electric field is formed between the first electrode assembly 610 and the second electrode assembly 620 through the fluid 608 along a first path in the first operational mode 602. FIG. 8 shows flux lines of the electric field in the first operational mode 602 between the first electrode assembly 610 and the second electrode assembly 620 along the first path. The capacitive sensor 600 is configured to measure a first capacitance between the first electrode assembly 610 and the second electrode assembly 620. The first capacitance may be utilized (e.g., by the controller 300, etc.) to determine a first permittivity of the fluid 608. For example, the controller 300 may determine the first permittivity of the fluid 608 utilizing the first capacitance and equations similar to the equations discussed above in relation to the first capacitive sensor 400 and the second capacitive sensor 450 and properties (e.g., geometry, material properties, properties of the excitement, etc.) of the capacitive sensor 600. In other embodiments, the second inner electrode 622 and the second outer electrode 624 are supplied with the excitement such that an electric field is formed between the second electrode assembly 620 and the first electrode assembly 610 through the fluid 608.

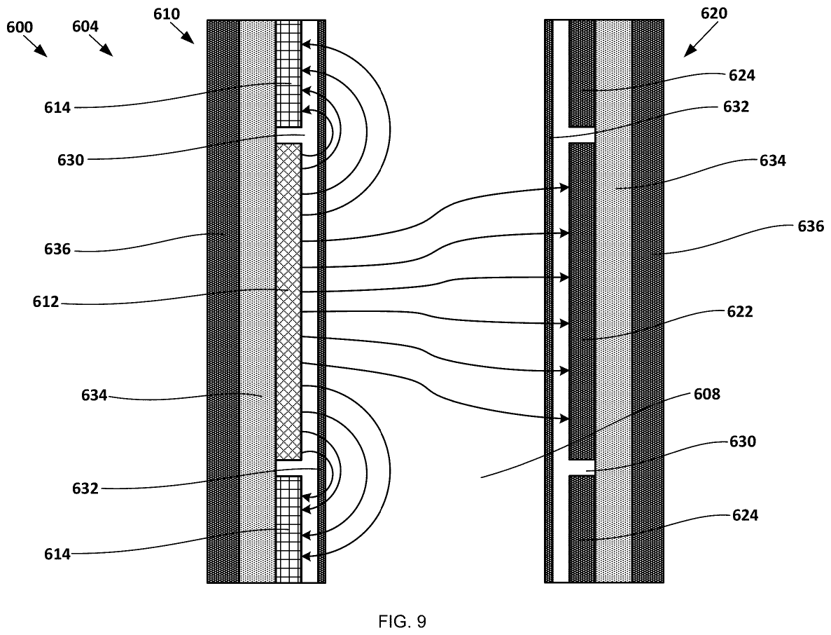

Referring now to FIG. 9, the second operational mode 604 of the capacitive sensor 600 is shown. In the second operational mode 604, the first inner electrode 612 is supplied with the excitement such that an electric field is formed between the first inner electrode 612 and the first outer electrode 614 through the fluid 608 along a second path. In some embodiments, a second electric field may be formed between the first inner electrode 612 and the second inner electrode 622 through the fluid 608 along the first path in the second operational mode 604. The capacitive sensor 600 is configured to measure a second capacitance between the first inner electrode 612 and the first outer electrode 614. The second capacitance may be utilized (e.g., by the controller 300, etc.) to determine a second permittivity of the fluid 608. For example, the controller 300 may determine the second permittivity of the fluid 608 utilizing the second capacitance and equations similar to the equations discussed above in relation to the first capacitive sensor 400 and the second capacitive sensor 450 and properties of the capacitive sensor 600. In other embodiments, the first outer electrode 614 is supplied with the excitement such that an electric field is formed between the first outer electrode 614 and the first inner electrode 612 through the fluid 608.

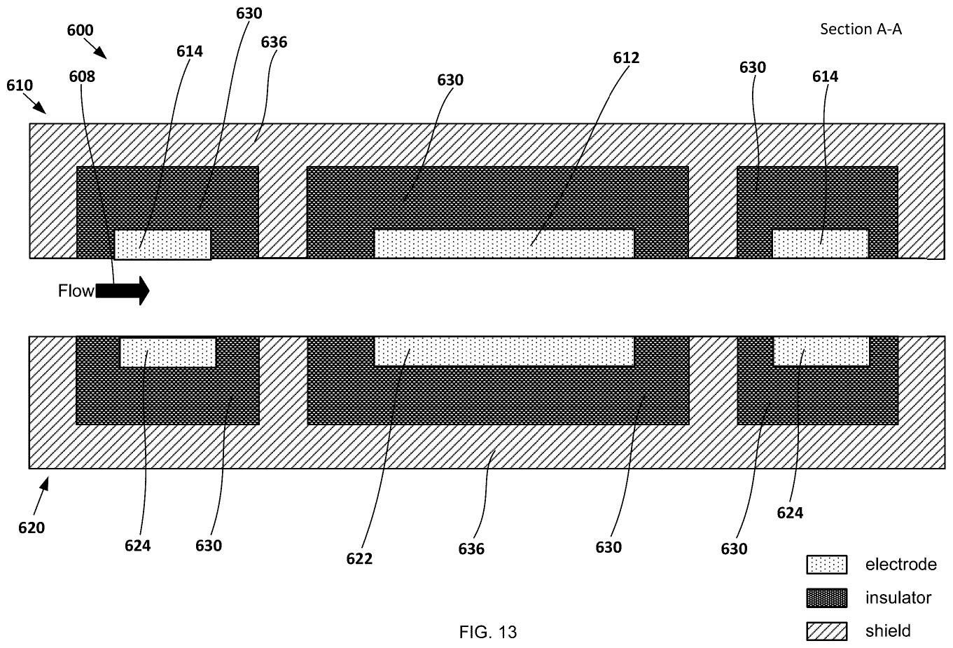

In the example embodiment shown in FIG. 13, the shield 636 extends between the first inner electrode 612 and the first outer electrode 614, which may block a path of the electric field through the insulator 630 such that a majority of the electric field extending between the first inner electrode 612 and the first outer electrode 614 in the second operational mode 604 extends through the fluid 608 instead of directly between the first inner electrode 612 and the first outer electrode 614 through the insulator 630. It may be desirable for the electric field between the first inner electrode 612 and the first outer electrode 614 to not extend through the insulator 630 positioned between the first inner electrode 612 and the first outer electrode 614 as the insulator capacitance can be multiple times higher than the capacitance of the fluid 608. Since the shortest distance between the first inner electrode 612 and the first outer electrode 614 is through the insulator 630 positioned between the first inner electrode 612 and the first outer electrode 614, this area of the insulator 630 can be the most sensitive area when measuring the second capacitance with the capacitive sensor 600 in the second operational mode 604. These factors can result in an insulator capacitance measured through the insulator 630 contributing significantly more to the second capacitance than a fluid capacitance measured through the fluid 608, resulting in sensitivity issues when trying to determine measurement influencing factors affecting the capacitive sensor 600 using the second capacitance. In the example embodiments shown in FIG. 13, the insulator 630 is also positioned between (i) each of the first inner electrode 612, the first outer electrode 614, the second inner electrode 622, and the second outer electrode 624 and (ii) the shield 636 such that the shield 636 does not directly contact the first inner electrode 612, the first outer electrode 614, the second inner electrode 622, or the second outer electrode 624. It may be desirable to isolate the first inner electrode 612, the first outer electrode 614, the second inner electrode 622, and the second outer electrode 624 from the shield 636 as the shield 636 may be conductive.

Referring back to FIG. 9, flux lines of the electric field in the second operational mode 604 are shown between the first inner electrode 612 and the first outer electrode 614 along the second path and flux lines of the second electric field between the first inner electrode 612 and the second inner electrode 622 along the first path. The flux lines directly between the first inner electrode 612 and the first outer electrode 614 through the insulator 630 are not shown as these flux lines may be neglected when the shield 636 is positioned between the first inner electrode 612 and the first outer electrode 614.

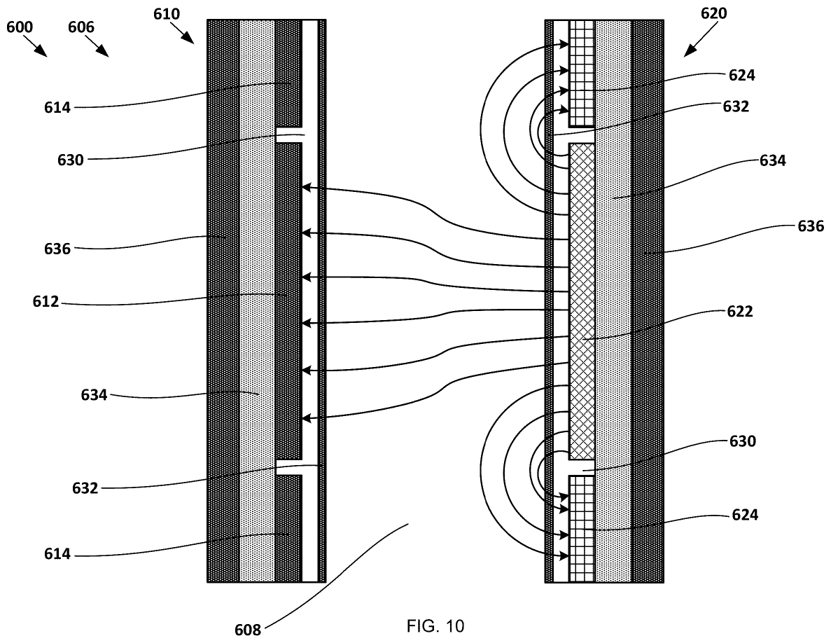

Referring now to FIG. 10, the third operational mode 606 of the capacitive sensor 600 is shown. In the third operational mode 606, the second inner electrode 622 is supplied with the excitement such that an electric field is formed between the second inner electrode 622 and the second outer electrode 624 through the fluid 608 along a third path. In some embodiments, a second electric field may be formed between the second inner electrode 622 and the first inner electrode 612 through the fluid 608 along the first path in the second operational mode 604. the capacitive sensor 600 is configured to measure a third capacitance between the second inner electrode 622 and the second outer electrode 624. The third capacitance may be utilized (e.g., by the controller 300, etc.) to determine a third permittivity of the fluid 608. For example, the controller 300 may determine the third permittivity of the fluid 608 utilizing the third capacitance and equations similar to the equations discussed above in relation to the first capacitive sensor 400 and the second capacitive sensor 450 and properties of the capacitive sensor 600. In other embodiments, the second outer electrode 624 is supplied with the excitement such that an electric field is formed between the second outer electrode 624 and the second inner electrode 622 through the fluid 608. In some embodiments, the shield 636 may extend between the second inner electrode 622 and the second outer electrode 624, which may block a path of the electric field through the insulator 630 such that a majority of the electric field extending between the second inner electrode 622 and the second outer electrode 624 in the third operational mode 606 extends through the fluid 608 instead of directly between the second inner electrode 622 and the second outer electrode 624 through the insulator 630.

Still referring to FIG. 10, flux lines of the electric field in the third operational mode 606 are shown between the second inner electrode 622 and the second outer electrode 624 along the third path and flux lines of the second electric field between the second inner electrode 622 and the first inner electrode 612 along the first path. The flux lines directly between the second inner electrode 622 and the second outer electrode 624 through the insulator 630 are not shown as these flux lines may be neglected when the shield 636 is positioned between the second inner electrode 622 and the second outer electrode 624.

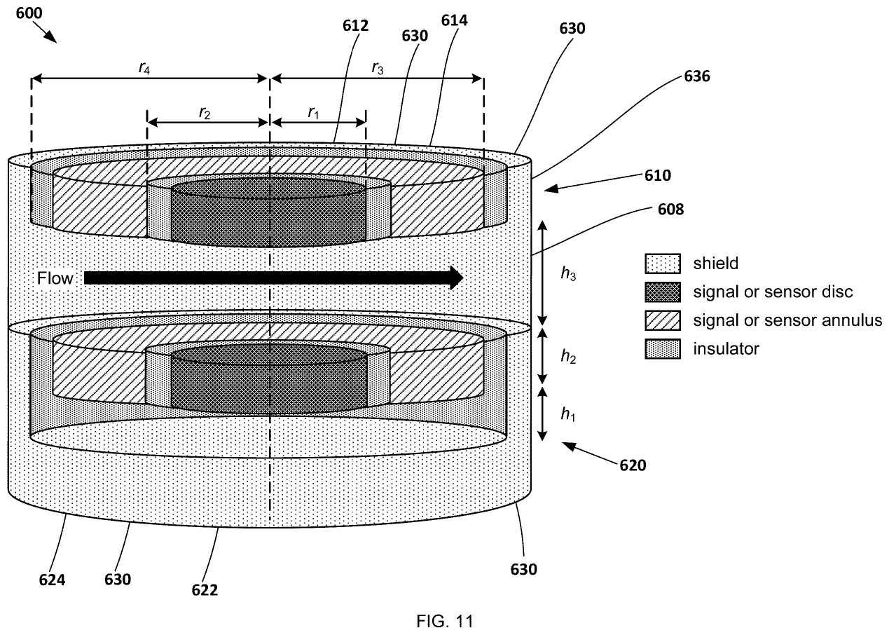

Referring now to FIG. 11, the capacitive sensor 600 is shown with dimensional indicators. The capacitance across the flow gap containing the fluid 608 between the first electrode assembly 610 and the second electrode assembly 620 may be determined using the equation:

$$C = ε A / {h}_{3}$$

where A is a combined cross-sectional area of the first inner electrode 612 and the first outer electrode 614 and the distance h₃ is an average distance between the first inner electrode 612 and the second inner electrode 622 (e.g., the distance across the flow gap containing the fluid 608, etc.). In some embodiments, the capacitance measured across the flow gap may be slightly increased due to additional flux outside of a volume of the flow gap positioned between the first outer electrode 614 and the second outer electrode 624. For example, the electric field between the first electrode assembly 610 and the second electrode assembly 620 may extend slightly beyond an outer diameter of the first outer electrode 614. In some embodiments, the controller 300 may be configured to compensate for this increase in capacitance due to the additional flux.

Erosion on the first electrode assembly 610 and the second electrode assembly 620 will have different impacts on the capacitances measured in the first operational mode 602, the second operational mode 604, and the third operational mode 606. Erosion on the first electrode assembly 610 or the second electrode assembly 620 will reduce the first capacitance measured by the capacitive sensor 600 in the first operational mode 602 due to a relative increase in the distance h₃ between the first electrode assembly 610 and the second electrode assembly 620, but the effect of the erosion on the second capacitance measured by the capacitive sensor 600 in the second operational mode 604 or the third capacitance measured by the capacitive sensor 600 in the third operational mode 606 will be negligible. In some embodiments, the second capacitance associated with the second operational mode 604 and the third capacitance associated with the third operational mode 606 may be effected by erosion if parts of the first electrode assembly 610 or the second electrode assembly 620 are completely eroded at edges (e.g., corners of elements of the first electrode assembly 610 or the second electrode assembly 620 are rounded by the erosion, etc.).

Deposited material (e.g., of solids suspended by the fluid 608, by chemical interactions between the capacitive sensor 600 and the fluid 608, etc.) on the first electrode assembly 610 and the second electrode assembly 620 will have different impacts on the capacitances measured in the first operational mode 602, the second operational mode 604, and the third operational mode 606. In some embodiments, deposits on the first electrode assembly 610 or the second electrode assembly 620 will only impact the capacitances measured in the first operational mode 602, the second operational mode 604, and the third operational mode 606 if a permittivity of the deposited material is different from the permittivity of the fluid 608. For example, if the permittivity of the deposited material is substantially the same as the permittivity of the fluid 608, the capacitances measured in the first operational mode 602, the second operational mode 604, and the third operational mode 606 will be substantially similar to the capacitances measured when there is not any of the deposited material.

If the permittivity of the deposited material is higher than the permittivity of the fluid 608, each of the first capacitance measured in the first operational mode 602, the second capacitance measured in the second operational mode 604, and the third capacitance measured in the third operational mode 606 will increase. However, the second capacitance measured in the second operational mode 604 and the third capacitance measured in the third operational mode 606 (e.g., the coplanar capacitances, the secondary capacitance measurements, etc.) will increase at a faster rate than the first capacitance measured in the first operational mode 602. If the permittivity of the deposited material is lower than the permittivity of the fluid 608, each of the first capacitance measured in the first operational mode 602, the second capacitance measured in the second operational mode 604, and the third capacitance measured in the third operational mode 606 will decrease. However, the second capacitance measured in the second operational mode 604 and the third capacitance measured in the third operational mode 606 will decrease at a faster rate than the first capacitance measured in the first operational mode 602.

For example, a surface deposit on the first electrode assembly 610 may result in an apparent change of the permittivity of the fluid in the first capacitance measured in the first operational mode 602 and the second capacitance measured in the second operational mode 604. The controller 300 may determine that the apparent change of the permittivity of the fluid in the first capacitance and the second capacitance is due to the surface deposit based on a change in the second capacitance being greater than a change in the first capacitance. In some embodiments, the controller 300 may be configured to recalibrate the capacitive sensor 600 to adjust the first capacitance and the second capacitance to remove the apparent change in order to accurately determine the permittivity of the fluid 608. In some embodiments, changes in the first capacitance measured in the first operational mode 602, the second capacitance measured in the second operational mode 604, and the third capacitance measured in the third operational mode 606 due to surface deposits may be proportional to a surface sensitivity of the capacitive sensor 600.

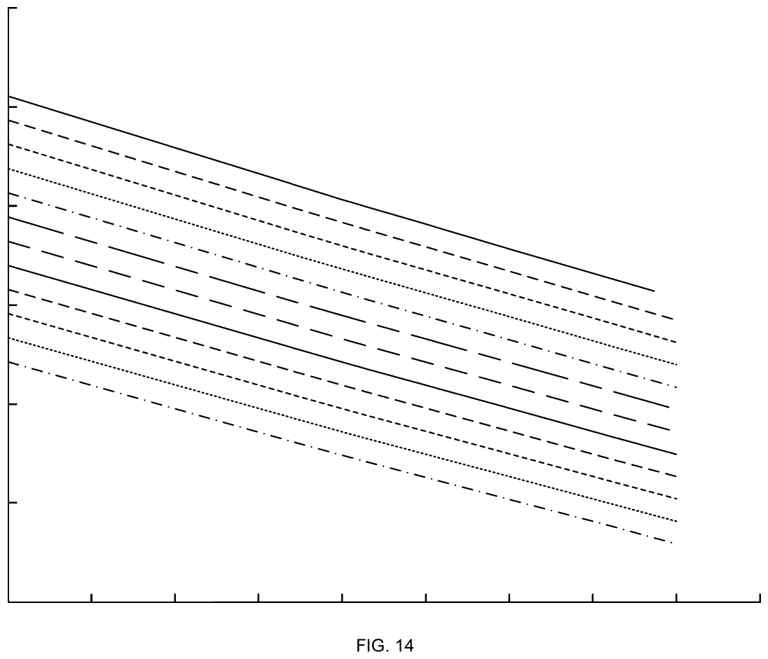

Referring to FIG. 14, an example graph of the effects of electrode wear on the first capacitance measured in the first operational mode 602 (e.g., parallel capacitance, capacitance measured between the first electrode assembly 610 and the second electrode assembly 620, etc.) are shown, where the y-axis represents capacitance and the x-axis represents an amount of erosion of the first electrode assembly 610 and/or the second electrode assembly 620. The y-axis and the x-axis of the example graph are unitless as the results are dependent on choices of the physical geometry of the first electrode assembly 610 and the second electrode assembly 620. As shown in FIG. 14, as the erosion increases, the capacitance decreases.

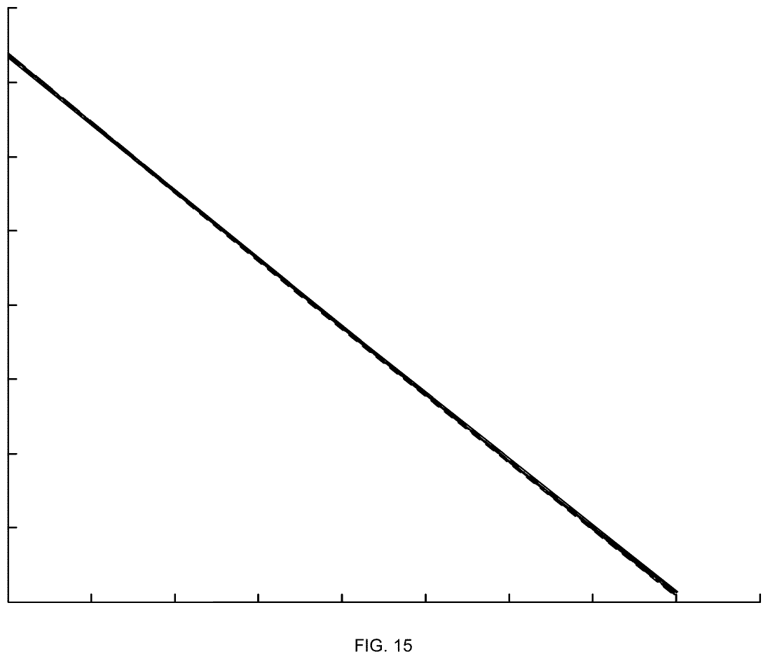

Referring to FIG. 15, an example graph of the effects of electrode wear on the second capacitance measured in the second operational mode 604 and/or the third capacitance measured in the third operational mode 606 are shown, where the y-axis represents capacitance and the x-axis represents an amount of erosion of the first electrode assembly 610 and/or the second electrode assembly 620. The y-axis and the x-axis of the example graph are unitless as the results are dependent on choices of the physical geometry of the first electrode assembly 610 and the second electrode assembly 620. As shown in FIG. 15, as the erosion increases, the capacitance decreases. However, as shown in FIGS. 14 and 15, the sensitivity of the decrease in the capacitance associated with the increase in the erosion is different between the first capacitance measured in the first operational mode 602 and the second capacitance measured in the second operational mode 604. The difference between the sensitivities may allow for an amount of erosion of the capacitive sensor 600 to be determined based on the relationship between the first capacitance measured in the first operational mode 602 and the second capacitance measured in the second operational mode 604.

The second capacitance measured in the second operational mode 604 and the third capacitance measured in the third operational mode 606 are not exhaustive for examples of secondary measurements of capacitance from the capacitive sensor 600 that may be used to verify and/or calibrate the first capacitance measured in the first operational mode 602. For example, additional secondary measurements may include a measurement of a capacitance between the first inner electrode 612 and the second inner electrode 622, a measurement of a capacitance between the first outer electrode 614 and the second outer electrode 624, a measurement of a capacitance between the first inner electrode 612 and the second outer electrode 624, a measurement of a capacitance between the first outer electrode 614 and the second inner electrode 622, etc. Further, electrodes that are unused in the measurements of capacitance described herein may be driven to different potentials to create guard electrodes (e.g., an electric shield for the capacitive sensor 600, etc.) that affect the electric fields used for the measurement of capacitance. For example, for the measurement of the capacitance between the first inner electrode 612 and the second inner electrode 622, the first outer electrode 614 and the second outer electrode 624 may be provided with an excitement to affect the electric field between the first inner electrode 612 and the second inner electrode 622 such that leakage of the electric field (e.g., an amount of the electric field that does not extend between the first inner electrode 612 and the second inner electrode 622, etc.) is minimized.

In various embodiments, the capacitive sensor 600 may be configured to measure capacitances with one driving electrode (e.g., one electrode provided with excitement, etc.) and three sensing electrodes (e.g., three electrodes configured to sense a flux from an electric field produced by the one driving electrode, etc.) such that the capacitive sensor 600 may measure three capacitances based on the excitement of one of the electrodes. For example, the capacitive sensor 600 may provide excitement to the first inner electrode 612 and the capacitive sensor 600 may measure capacitances between the first inner electrode 612 and the first outer electrode 614, between the first inner electrode 612 and the second inner electrode 622, and between the first inner electrode 612 and the second outer electrode 624. In various embodiments, the capacitive sensor 600 may be configured to measure capacitances with three driving electrodes (e.g., three electrodes provided with excitement, etc.) and one sensing electrode (e.g., one electrode configured to sense fluxes from electric fields produced by the three driving electrodes, etc.) such that the capacitive sensor 600 may measure three capacitances based on the excitement of three of the electrodes. For example, the capacitive sensor 600 may provide excitement to the first inner electrode 612, the first outer electrode 614, and the second outer electrode 624 and the capacitive sensor may measure capacitances between the first inner electrode 612 and the second inner electrode 622, between the first outer electrode 614 and the second inner electrode 622, and between the second outer electrode 624 and the second inner electrode 622. In various embodiments, the capacitive sensor 600 may be configured to measure capacitances between a various number of driving electrodes (e.g., one driving electrode, two driving electrodes, three driving electrode, four driving electrodes, etc.) and a various number of sensing electrodes (e.g., one sensing electrode, two sensing electrodes, three sensing electrodes, four sensing electrodes, etc.) such that the capacitive sensor 600 may measure a various number of capacitances (e.g., one capacitance, two capacitances, three capacitances, four capacitances, five capacitances, etc.) between each of the driving electrodes and each of the sensing electrodes.

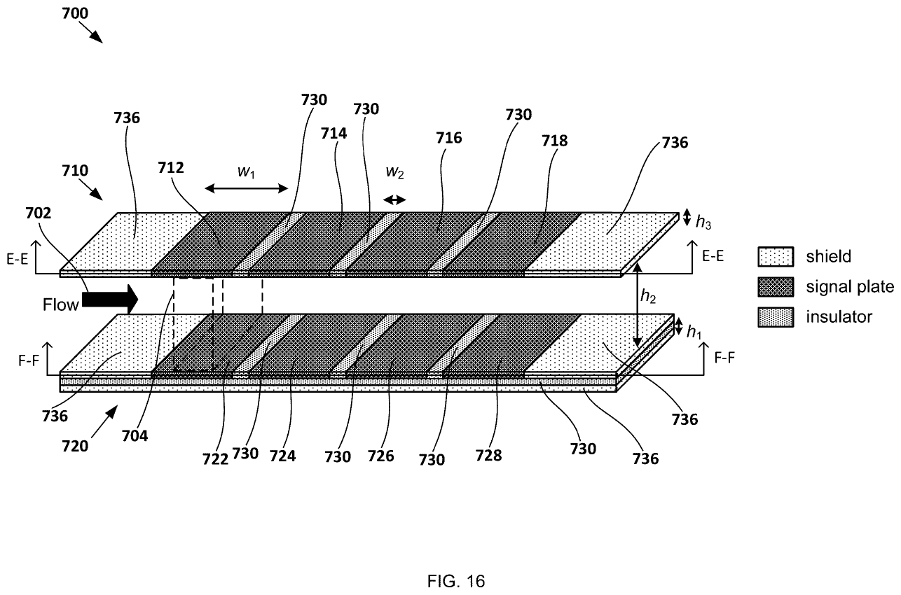

Referring to FIGS. 16-19, a second example embodiment of a capacitive sensor 700 is shown. In some embodiments, the capacitive sensor 210 may be configured as the capacitive sensor 700. The capacitive sensor 700 may be operated in multiple operational modes similar to capacitive sensor 600 described herein and is configured to measure a capacitance associated with a fluid 702. The fluid 702 may be flowing through the capacitive sensor 700 and/or may be the fluid 16 flowing through the pipeline 12. In some embodiments, the fluid 702 may be modeled as one or more fluid slugs 704 (e.g., fluid slugs 18, etc.). For example, the fluid slug 704 can represent a certain amount, volume, portion, or quantity of the fluid 702.

The capacitive sensor 700 includes a first electrode assembly 710 and a second electrode assembly 620 separated by a fluid-filled gap filled by the fluid 702. In some embodiments, the capacitive sensor 700 may be configured to be installed as part of the pipeline 12. For example, the first electrode assembly 710 may be configured to be installed on a first side of the pipeline 12 and the second electrode assembly 720 may be configured to be installed on a second side of the pipeline 12 radially opposite the first electrode assembly 710 such that the first electrode assembly 710 and the second electrode assembly 720 are positioned on opposite sides of the fluid 16 flowing through the pipeline 12.

The first electrode assembly 710 is configured as a first co-planar capacitor that includes a first electrode 712, a second electrode 714, a third electrode 716, and a fourth electrode 718, according to some embodiments. According to the example embodiment shown in FIG. 16, the first electrode 712 defines a first electrode plane D-D through the first electrode 712. The second electrode 714 is positioned downstream (e.g., in a direction of a flow of the fluid 702, etc.) of the first electrode 712 on the first electrode plane D-D. The third electrode 716 is positioned downstream of the second electrode 714 on the first electrode plane D-D. The fourth electrode 718 is positioned downstream of the third electrode 716 on the first electrode plane D-D.

The second electrode assembly 720 is configured as a second co-planar capacitor that includes a fifth electrode 722, a sixth electrode 724, a seventh electrode 726, and a eighth electrode 728, according to some embodiments. According to the example embodiment shown in FIG. 16, the fifth electrode 722 defines a second electrode plane E-E through the fifth electrode 722. The sixth electrode 724 is positioned downstream of the fifth electrode 722 on the second electrode plane E-E. The seventh electrode 726 is positioned downstream of the sixth electrode on the second electrode plane E-E. The eighth electrode 728 is positioned downstream of the seventh electrode 726 on the second electrode plane E-E.

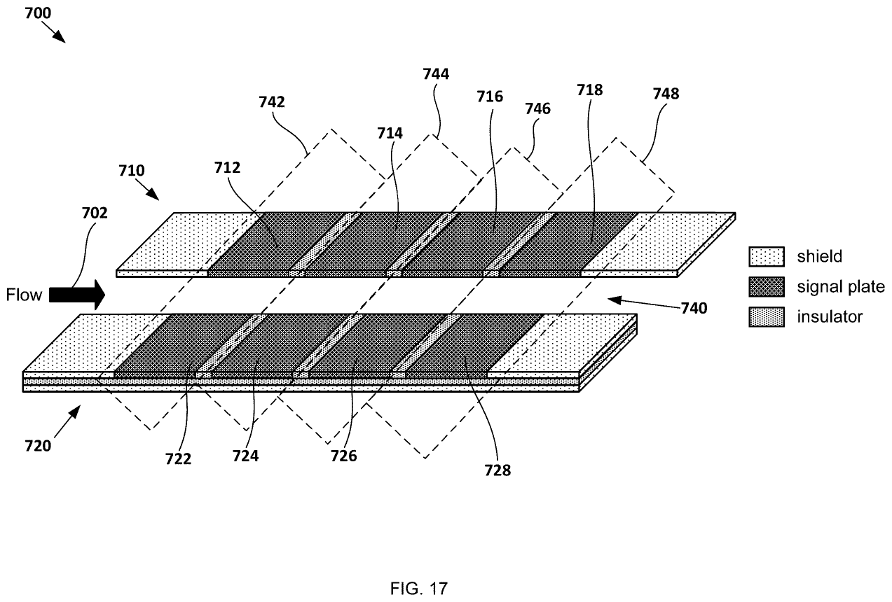

Referring now to FIG. 17, the electrodes of the first electrode assembly 710 and the electrodes of the second electrode assembly 720 may form a plurality of electrode pairs 740, according to some embodiments. For example, each of the electrode pairs 740 may be positioned on opposing sides of the fluid-filled gap containing the fluid 702. A first of the electrode pairs, shown as first electrode pair 742, includes the first electrode 712 of the first electrode assembly 710 and the fifth electrode 722 of the second electrode assembly 720. A second of the electrode pairs, shown as second electrode pair 744, includes the second electrode 714 of the first electrode assembly 710 and the sixth electrode 724 of the second electrode assembly 720. A third of the electrode pairs, shown as third electrode pair 746, includes the third electrode 716 of the first electrode assembly 710 and the seventh electrode 726 of the second electrode assembly 720. A fourth of the electrode pairs, shown as fourth electrode pair 748, includes the fourth electrode 718 of the first electrode assembly 710 and the eighth electrode 728 of the second electrode assembly 720.

In some embodiments, the capacitive sensor 700 is configured to measure a capacitance between each of electrode pairs 740. For example, the capacitive sensor 700 may be configured to measure a first capacitance between the first electrode pair 742, a second capacitance between the second electrode pair 744, a third capacitance between the third electrode pair 746, and a fourth capacitance between the fourth electrode pair. In some embodiments, the capacitance measured between each of the electrode pairs 740 may be considered a primary capacitance measurement. For example, the capacitance measured between each of the electrode pairs 740 may be the capacitance measurement utilized by the controller 300 to make decisions regarding the operation of the pipeline 12.

In some embodiments, the controller 300 may utilize the capacitances measured between each of the electrode pairs 740 to determine a velocity (e.g., an initial velocity, a pre-calibration velocity, etc.) of the flow of the fluid 702. For example, the first electrode pair 742 may measure the first capacitance through the fluid slug 704 (e.g., a first fluid parcel, etc.) of the fluid 702 at a first time and the second electrode pair 744 may measure the second capacitance through the fluid slug 704 of the fluid 702 at a second time. If a first permittivity of the fluid 702 corresponding with the first capacitance is substantially equal (e.g., within 1%, within 2%, within 5%, within 10%, etc.) to a second permittivity of the fluid 702 corresponding with the second capacitance, it may be an indication that the fluid slug 704 has traveled between the first electrode pair 742 and the second electrode pair 744 between the first time and the second time. Utilizing positional relationships (e.g., a distance between components, etc.) of the capacitive sensor 700, the controller 300 may determine the velocity of the flow of the fluid 702.

Additionally, the controller 300 the third capacitance measured by the third electrode pair 746 may be utilized to verify the velocity of the flow of the fluid. For example, the controller 300 may determine a third time that corresponds with when the fluid slug 704 will be positioned proximate the third electrode pair 746 using the velocity of the flow of the fluid 702 and the positional relationships of the capacitive sensor 700. If a third permittivity of the fluid 702 corresponding with the third capacitance measured by the third electrode pair 746 at the third time is substantially equal to the first permittivity corresponding with first capacitance and the second permittivity corresponding with the second capacitance, then the measurement of the first electrode pair 742, the measurement of the second electrode pair 744, the measurement of the third electrode pair, and the velocity may be considered verified. If the third permittivity corresponding with the third capacitance measured by the third electrode pair 746 at the third time is not substantially equal to the first permittivity corresponding with first capacitance and the second permittivity corresponding with the second capacitance, then the measurement of the first electrode pair 742, the measurement of the second electrode pair 744, the measurement of the third electrode pair, and the velocity may be considered unverified and further verification may be performed (e.g., to determine the sensor error of the capacitive sensor 700, provide the sensor error to a user via a display device, etc.) and/or the controller 300 may calibrate the capacitive sensor 700.

In some embodiments, the controller 300 may be configured to recalculate a calibrated velocity of the fluid 702 in response to the velocity being considered unverified (e.g., recalculate the calibrated velocity of the fluid 702 after calibrating the capacitive sensor 700, etc.). For example, after the controller 300 has calibrated the capacitive sensor 700, the first electrode pair 742 may measure the first capacitance (e.g., a first calibrated capacitance, etc.) of the fluid slug 704 (e.g., a second fluid parcel, etc.) of the fluid at a fourth time and the second electrode pair 744 may measure the second capacitance (e.g., a second calibrated capacitance, etc.) of the fluid slug 704 of the fluid 702 at a fifth time. If a fourth permittivity of the fluid 16 (e.g., a first calibrated value of a fluid property, etc.) corresponding with the first capacitance through the fluid slug 704 measured at the fourth time is substantially equal to a fifth permittivity of the fluid 16 (e.g., a second calibrated value of a fluid property, etc. corresponding with the second capacitance through the fluid slug 704 measured at the fifth time, it may be an indication that the fluid slug 704 has traveled between the first electrode pair 742 and the second electrode pair 744 between the fourth time and the fifth time. Utilizing positional relationships (e.g., a distance between components, etc.) of the capacitive sensor 700, the controller 300 may determine the calibrated velocity of the flow of the fluid 702. The controller 300 may provide the calibrated velocity to the user interface 320.

According to various embodiments, the capacitive sensor 700 is configured to measure a capacitance between electrodes of the first electrode assembly 710. For example, the capacitive sensor 700 may be configured to measure a fifth capacitance between the first electrode 712 and the second electrode 714, a sixth capacitance between the second electrode 714 and the third electrode 716, and a seventh capacitance between the third electrode 716 and the fourth electrode 718. In some embodiments, the capacitance measured between the electrodes of the first electrode assembly 710 may be considered a secondary capacitance measurement. For example, the capacitance measured between the electrodes of the first electrode assembly 710 may be the capacitance measurements utilized by the controller 300 to verify and calibrate the primary capacitance measurements measured between the electrode pairs 740. In some embodiments, the controller 300 may utilize a capacitance measurement between the electrodes of the first electrode assembly 710 to determine a sensor error associated with capacitances measured between the electrode pairs 740. For example, a permittivity (e.g., a fourth value of a fluid property, etc.) corresponding with the capacitance measurement between the electrodes of the first electrode assembly 710 may be modeled with the permittivities corresponding with the capacitances measured between the electrode pairs 740 to determine the sensor error.

According to various embodiments, the capacitive sensor 700 is configured to measure a capacitance between electrodes of the second electrode assembly 720. For example, the capacitive sensor 700 may be configured to measure an eighth capacitance between the fifth electrode 722 and the sixth electrode 724, a ninth capacitance between the sixth electrode 724 and the seventh electrode 726, and a tenth capacitance between the seventh electrode 726 and the eighth electrode 728. In some embodiments, the capacitance measured between the electrodes of the second electrode assembly 720 may be considered a secondary capacitance measurement. For example, the capacitance measured between the electrodes of the second electrode assembly 720 may be the capacitance measurements utilized by the controller 300 to verify and calibrate the primary capacitance measurements measured between the electrode pairs 740. In some embodiments, the controller 300 may utilize a capacitance measurement between the electrodes of the second electrode assembly 720 to determine a sensor error associated with capacitances measured between the electrode pairs 740. For example, a permittivity corresponding with the capacitance measurement between the electrodes of the second electrode assembly 720 may be modeled with the permittivities corresponding with the capacitances measured between the electrode pairs 740 to determine the sensor error.

Referring back to FIG. 16, the first electrode assembly 710 and the second electrode assembly 720 also include an insulator 730, according to some embodiments. For the first electrode assembly 710, the insulator 730 is positioned between each of the adjacent electrodes of the first electrode assembly 710 (e.g., between the first electrode 712 and the second electrode 714, between the second electrode 714 and the third electrode 716, etc.) and is configured to electrically insulate each of the adjacent electrodes of the first electrode assembly 710. For the second electrode assembly 720, the insulator 730 is positioned between each of the adjacent electrodes of the second electrode assembly 720 (e.g., between the fifth electrode 722 and the sixth electrode 724, between the sixth electrode 724 and the seventh electrode 726, etc.) and is configured to electrically insulate each of the adjacent electrodes of the second electrode assembly 720. In some embodiments, the insulator 730 is also positioned between (i) the electrodes of the first electrode assembly 710 and the electrodes of the second electrode assembly 720 and (ii) the fluid 702 to electrically isolate the fluid 702 from the electrodes. In some embodiments, the first electrode assembly 710 and the second electrode assembly 720 also include an inner sheath positioned along an inside surface of the first electrode assembly 710 and the second electrode assembly 720 configured to contact the fluid 702 that is similar to the inner sheath 632 of the capacitive sensor 600. In some embodiments, the first electrode assembly 710 and the second electrode assembly 720 also include a control layer configured to selectively provide electrical excitement to the electrodes of the first electrode assembly 710 and/or the electrodes of the second electrode assembly 720 similar to the control layer 634 of the capacitive sensor 600.

According to the example embodiment shown in FIGS. 16-19, the first electrode assembly 710 and the second electrode assembly 720 also include a shield 736. The shield 736 is configured to block electrical fields produced between the electrodes of the first electrode assembly 710 and/or the electrodes of the second electrode assembly 720. In some embodiments, the shield 736 may be configured as a ground. In some embodiments, the shield 736 is configured to protect an outside surface of the first electrode assembly 710 and the second electrode assembly 720 that is opposite of the inside surface of the first electrode assembly 710 and the second electrode assembly 720. The shield 736 may be configured to protect the first electrode assembly 710 and the second electrode assembly 720 from objects coming in contact with the first electrode assembly 710 and the second electrode assembly 720 and damaging the first electrode assembly 710 and the second electrode assembly 720. For example, the shield 736 may be a layer of metal configured to resist damage. In other embodiments, the shield 736 may be formed of a rubber or a plastic.

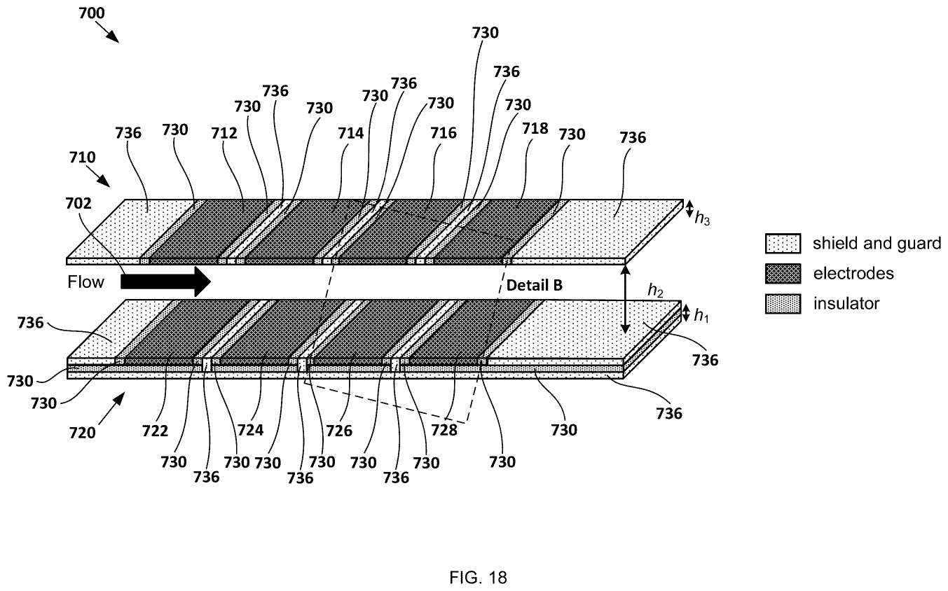

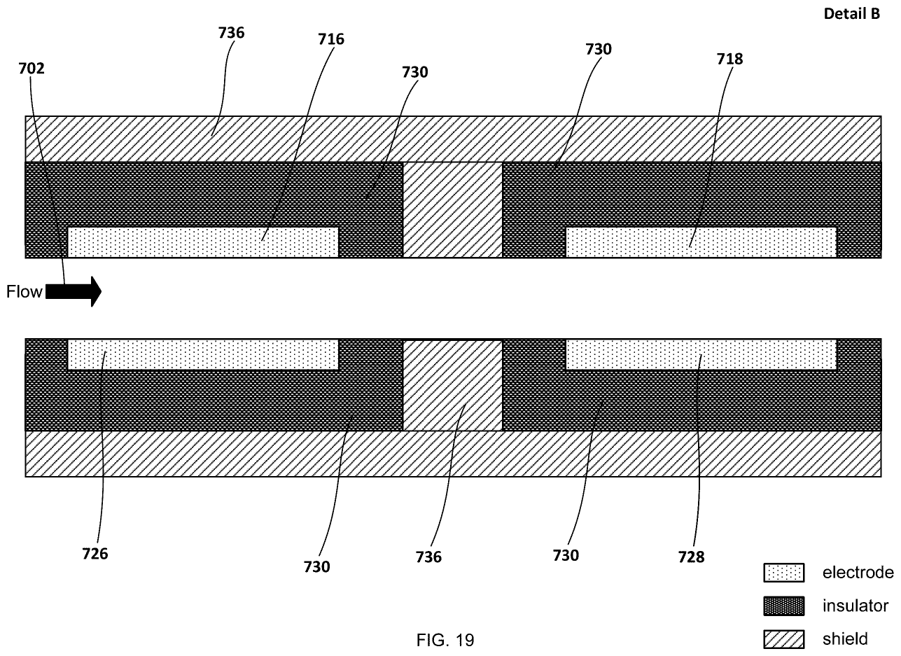

According to the example embodiment shown in FIGS. 18 and 19, the shield 736 is also positioned between the adjacent electrodes of the first electrode assembly 710 and/or the adjacent electrodes of the second electrode assembly 720. For example, the shield 736 may be positioned in between each of the adjacent electrodes of the first electrode assembly 710 (e.g., between the first electrode 712 and the second electrode 714, between the second electrode 714 and the third electrode 716, etc.) and is configured to block the flux between each of the adjacent electrodes of the first electrode assembly 710 through the shield 736 (e.g., the flux that does not pass through the fluid 702, etc.). As another example, the shield 736 may be positioned in between each of the adjacent electrodes of the second electrode assembly 720 (e.g., between the fifth electrode 722 and the sixth electrode 724, between the sixth electrode 724 and the seventh electrode 726, etc.) and is configured to block the flux between each of the adjacent electrodes of the second electrode assembly 720 through the shield 736. In some embodiments, the shield 736 may be continuous. In embodiments, the shield 736 may be formed of several separate elements.

### Processes

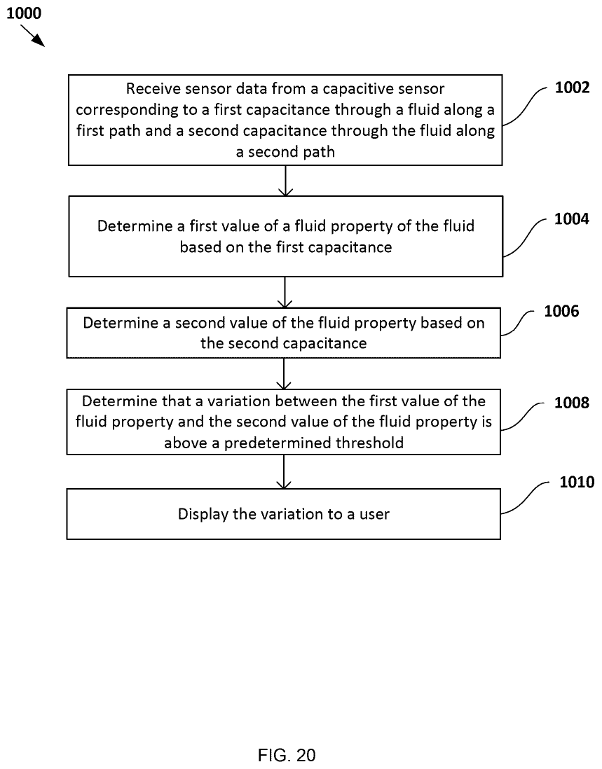

Referring now to FIG. 20, a flow diagram of a process 1000 (e.g., a method, etc.) for validating a capacitive sensor is shown, according to some embodiments. Process 1000 includes steps 1002-1012 and can be performed by the system 10, the controller 300, and/or the control system 100, according to some embodiments. In some embodiments, process 1000 is performed in order to calibrate the capacitive sensor in order to reduce errors in measurements of the capacitive sensor.

The process 1000 includes receiving sensor data from a capacitive sensor corresponding to a first capacitance through a fluid along a first path and a second capacitance through the fluid along a second path (step 1002), according to some embodiments. In some embodiments, step 1002 includes receiving an input indicating that the process 1000 should be initiated. In some embodiments, the process 1000 is automatically initiated based on user inputs or operational conditions (e.g., a flow of the fluid 16 through the pipeline 12, etc.). In some embodiments, the process 1000 receives the sensor data from the capacitive sensor 210, the capacitive sensor 500, the capacitive sensor 600, and/or the capacitive sensor 700.

The process 1000 includes determining a first value of a fluid property of the fluid based on the first capacitance (step 1004), according to some embodiments. In some embodiments, the fluid property is permittivity and the first value of the fluid property is a first permittivity of the fluid. In some embodiments, the first value of the fluid property is determined based on physical relationships of the capacitive sensor and/or known fluid properties that correspond to the first capacitance. In some embodiments, step 1004 is performed by the pipeline manager 312 of the controller 300 utilizing the sensor data from the capacitive sensor 210 and data from the database 310.

The process 1000 includes determining a second value of a fluid property of the fluid based on the second capacitance (step 1006), according to some embodiments. In some embodiments, the fluid property is permittivity and the second value of the fluid property is a second permittivity of the fluid. In some embodiments, the second value of the fluid property is determined based on physical relationships of the capacitive sensor and/or known fluid properties that correspond to the second capacitance. In some embodiments, step 1006 is performed by the pipeline manager 312 of the controller 300 utilizing the sensor data from the capacitive sensor 210 and data from the database 310.

The process 1000 includes determining that a variation between the first value of the fluid property and the second value of the fluid property is above a predetermined threshold (step 1008), according to some embodiments. In some embodiments, the predetermined threshold may be a calibration threshold set by an operator to determine when the capacitive sensor is out of calibration. In some embodiments, step 1008 is performed by the calibration manager 314 of the controller 300 utilizing the sensor data from the capacitive sensor 210 and data from the database 310. In some embodiments, step 1008 includes calibrating the capacitive sensor based on the variation between the first value of the fluid property and the second value of the fluid property.

The process 1000 includes displaying the variation to a user (step 1010), according to some embodiments. In some embodiments, step 1010 includes operating the user interface 320 to provide display data of the variation. The display data of the variation may include elements associated with the variation. In some embodiments, the elements associated with the variation are displayed to the user. For example, the elements may include a bar graph displaying the variation between the first value of the fluid property and the second value of the fluid property. In some embodiments, step 1010 is performed by the user interface 320.

In some embodiments, process 1000 includes receiving the sensor data from the capacitive sensor corresponding to a third capacitance through the fluid along a third path and the process 1000 further includes determining a third value of the fluid property of the fluid based on the third capacitance. In some embodiments, the fluid property is permittivity and the third value of the fluid property is a third permittivity of the fluid. In some embodiments, the third value of the fluid property is determined based on physical relationships of the capacitive sensor and/or known fluid properties that correspond to the third capacitance. In some embodiments, the third value of the fluid property is determined by the pipeline manager 312 of the controller 300 utilizing the sensor data from the capacitive sensor 210 and data from the database 310.

In some embodiments, process 1000 further includes modeling the first value of the fluid property with the second value of the fluid property and the third value of the fluid property to determine a sensor error. In some embodiments, the sensor error is associated with at least one of the first value of the fluid property, the second value of the fluid property or the third value of the fluid property. For example, the capacitive sensor may measure a first capacitance along a first path through a fluid, a second capacitance along a second path through the fluid, and a third capacitance along a third path through the fluid. Based on the first capacitance, the second capacitance, and the third capacitance, a first permittivity of the fluid corresponding to the first capacitance, a second permittivity of the fluid corresponding to the second capacitance, and a third permittivity of the fluid corresponding to the third capacitance may be determined. If the first permittivity and the second permittivity are substantially equal, then the sensor error may be associated with the third permittivity. If the second permittivity and the third permittivity are substantially equal, then the sensor error may be associated with the third permittivity. If none of the first permittivity, the second permittivity, or the third permittivity are substantially equal, then additional data may be required in order to determine the association of the sensor error (e.g., a measurement of a fourth capacitance in order to determine a fourth permittivity, a measurement of a fifth capacitance in order to determine a fifth permittivity, etc.). In some embodiments, the sensor error may be determined by comparing trends in the sensor data over time. For example, if a first permittivity corresponding to a first capacitance changes at a faster rate than a second permittivity corresponding to a second capacitance, then the sensor error may be associated with the first permittivity or the second permittivity depending on the circumstances.

In some embodiments, the process 1000 further includes displaying the sensor error to the user. In some embodiments, the user interface 320 is operated to provide display data of the sensor error. The display data of the sensor error may include elements associated with the sensor error. In some embodiments, the elements associated with the sensor error may include an identifier indicated a source of the sensor error (e.g., the measurement of the first capacitance, etc.). In some embodiments, the sensor error is displayed to the user in response to the sensor error being above a predetermined error threshold. The predetermined error threshold may be configured in order to identify if the capacitive sensor requires calibration. For example, the predetermined error threshold may be a value of the sensor error that corresponds to a requirement for calibration of the capacitive sensor.

In some embodiments, the process 1000 further includes calibrating the capacitive sensor. In some embodiments, the capacitive sensor is calibrated in response to the sensor error being above the predetermined error threshold. The calibration of the capacitive sensor may decrease the sensor error such that the sensor error is below the predetermined error threshold. For example, the calibration of the capacitive sensor may include filtering the sensor data provided by the capacitive sensor in order to decrease the sensor error.

In some embodiments, the process 1000 further includes determining a location of a deposit of a material on a surface of the capacitive sensor based on the sensor error. For example, the location of the deposit of the material on the surface of the capacitive sensor may be determined by comparing the permittivities corresponding to capacitances of the fluid measured by the capacitive sensor. In some embodiments, the process 1000 further includes determining a location of erosion on the surface of the capacitive sensor based on the sensor error.

In some embodiments, the process 1000 further includes displaying the location of the deposit of the material on the surface of the capacitive sensor or the location of the erosion on the surface of the capacitive sensor to the user. In some embodiments, the user interface 320 is operated to provide display data of the location of the deposit of the material on the surface of the capacitive sensor or the location of the erosion on the surface of the capacitive sensor to the user. The display data of the location of the deposit of the material on the surface of the capacitive sensor or the location of the erosion on the surface of the capacitive sensor may include elements. For example, the display data may include a graphical representation of the capacitive sensor that includes an indication of the location of the deposit of the material on the surface of the capacitive sensor or the location of the erosion on the surface of the capacitive sensor.

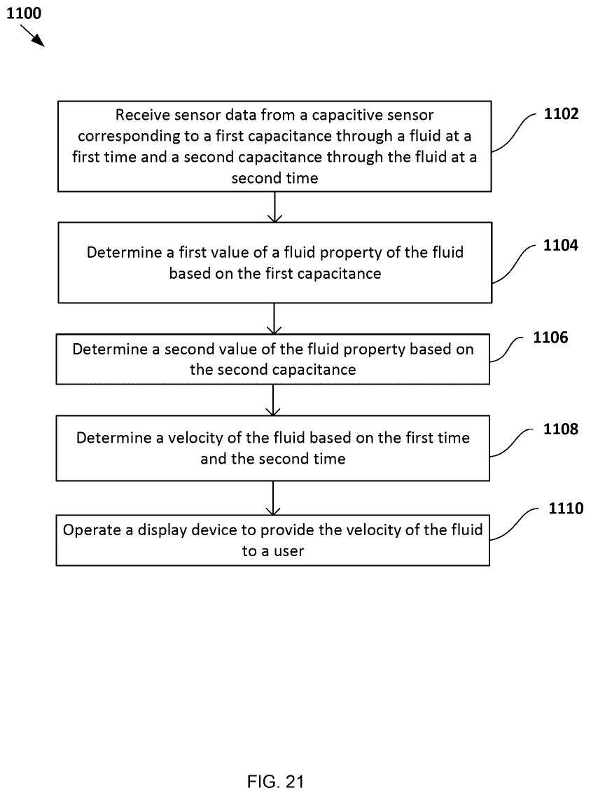

Referring now to FIG. 21, a flow diagram of a process 1100 (e.g., a method, etc.) for determining a velocity of a fluid is shown, according to some embodiments. The process 1100 includes steps 1102-1110 and can be performed by the system 10, the controller 300, and/or the control system 100, according to some embodiments. In some embodiments, the process 1100 is performed in order to calibrate a capacitive sensor to reduce errors in measurements of the capacitive sensor.

The process 1100 includes receiving sensor data from a capacitive sensor corresponding to a first capacitance through a fluid at a first time and a second capacitance through the fluid at a second time (step 1102), according to some embodiments. In some embodiments, step 1102 includes receiving an input indicating that the process 1100 should be initiated. In some embodiments, the process 1100 is automatically initiated based on user inputs or operational conditions (e.g., a flow of the fluid 16 through the pipeline 12, etc.). In some embodiments, the capacitive sensor includes a plurality of electrode pairs. Each of the electrode pairs are configured to measure a capacitance along a path between a first electrode and a second electrode of each of the electrode pairs. The path may be through the fluid flowing through a conduit. In some embodiments, the process 1100 includes receiving the sensor data from the capacitive sensor 210, the capacitive sensor 500, the capacitive sensor 600, and/or the capacitive sensor 700. In some embodiments, the first capacitance through the fluid and the second capacitance through the fluid are of a fluid parcel (e.g., a fluid slug, a first fluid parcel etc.) of the fluid.

The process 1100 includes determining a first value of a fluid property of the fluid based on the first capacitance (step 1104), according to some embodiments. In some embodiments, the fluid property is permittivity and the first value of the fluid property is a first permittivity of the fluid. In some embodiments, the first value of the fluid property is determined based on physical relationships of the capacitive sensor and/or known fluid properties that correspond to the first capacitance. In some embodiments, step 1104 is performed by the pipeline manager 312 of the controller 300 utilizing the sensor data from the capacitive sensor 210 and data from the database 310.

The process 1100 includes determining a second value of a fluid property of the fluid based on the second capacitance (step 1106), according to some embodiments. In some embodiments, the fluid property is permittivity and the second value of the fluid property is a second permittivity of the fluid. In some embodiments, the second value of the fluid property is determined based on physical relationships of the capacitive sensor and/or known fluid properties that correspond to the second capacitance. In some embodiments, step 1106 is performed by the pipeline manager 312 of the controller 300 utilizing the sensor data from the capacitive sensor 210 and data from the database 310. In some embodiments, the second value of the fluid property is substantially equal to the first value of the fluid property.

The process 1100 includes determining a velocity of the fluid based on the first time and the second time (step 1108), according to some embodiments. In some embodiments, the velocity of the fluid may be determined using physical relationships of the calibration sensor. For example, if the first capacitance is measured at a first location and the second capacitance is measured at a second location that is a distance away from the first location, the velocity of the fluid may be determined based on a difference between the first time and the second time and the distance between the first location and the second location. In some embodiments, step 1008 is performed by the pipeline manager 312 of the controller 300 utilizing the sensor data from the capacitive sensor 210 and data from the database 310.

The process 1100 includes displaying the velocity of the fluid to a user (step 1110), according to some embodiments. In some embodiments, step 1110 includes operating the user interface 320 to provide display data of the velocity. The display data of the velocity may include elements associated with the velocity. In some embodiments, the elements associated with the velocity are displayed to the user. For example, the elements may include an illustration of the velocity of the fluid. In some embodiments, step 1110 is performed by the user interface 320.

In some embodiments, the process 1100 includes determining a third time when the fluid parcel will be positioned near a component of the capacitive sensor configured to measure a third capacitance through the fluid. In some embodiments, the third time may be determined based on physical relationships of the capacitive sensor and the velocity of the fluid. In some embodiments, the third time is determined by the pipeline manager 312 of the controller 300 utilizing the sensor data from the capacitive sensor 210 and data from the database 310. In some embodiments, the process 1100 further includes receiving the sensor data from the capacitive sensor corresponding to a third capacitance through the fluid parcel of the fluid at the third time. In some embodiments, the sensor data is received from the capacitive sensor 210, the capacitive sensor 500, the capacitive sensor 600, and/or the capacitive sensor 700. In some embodiments, the third capacitance may be received from a third of the electrode pairs of the capacitive sensor.

In some embodiments, the process 1100 includes determining a third value of the fluid property of the fluid based on the third capacitance. In some embodiments, the fluid property is permittivity and the third value of the fluid property is a third permittivity of the fluid. In some embodiments, the third value of the fluid property is determined based on physical relationships of the capacitive sensor and/or known fluid properties that correspond to the third capacitance. In some embodiments, the third capacitance is determined by the pipeline manager 312 of the controller 300 utilizing the sensor data from the capacitive sensor 210 and data from the database 310.

In some embodiments, the process 1100 includes determining that a variation between the first value of the fluid property and the third value of the fluid property is above a predetermined threshold. In some embodiments, the predetermined threshold may be a calibration threshold set by an operator to determine when the capacitive sensor is out of calibration. The variation is determined by the calibration manager 314 of the controller 300 utilizing the sensor data from the capacitive sensor 210 and data from the database 310.

In some embodiments, the process 1100 further includes displaying the variation to the user. In some embodiments, the user interface 320 is operated to provide display data of the variation. The display data of the sensor error may include elements associated with the variation. In some embodiments, the elements associated with the variation may include an identifier indicated a source of the variation (e.g., the measurement of the first capacitance, etc.). In some embodiments, the variation is displayed to the user in response to the variation being above the predetermined threshold.

In some embodiments, the process 1100 includes receiving the sensor data from the capacitive sensor corresponding to a fourth capacitance through the fluid parcel. In some embodiments, the fourth capacitance through the fluid parcel is received from the first electrode of the first of the electrode pairs and the first electrode of the second of the electrode pairs. For example, the first electrode of the first of the electrode pairs and the first electrode of the second of the electrode pairs may be co-planar and the fourth capacitance may be a capacitance through the fluid between the first electrode of the first of the electrode pairs and the first electrode of the second of the electrode pairs. In some embodiments, the sensor data is received from the capacitive sensor 210, the capacitive sensor 500, the capacitive sensor 600, and/or the capacitive sensor 700.

In some embodiments, the process 1100 includes determining a fourth value of the fluid property of the fluid based on the fourth capacitance. In some embodiments, the fluid property is permittivity and the fourth value of the fluid property is a fourth permittivity of the fluid. In some embodiments, the fourth value of the fluid property is determined based on physical relationships of the capacitive sensor and/or known fluid properties that correspond to the fourth capacitance. In some embodiments, the fourth capacitance is determined by the pipeline manager 312 of the controller 300 utilizing the sensor data from the capacitive sensor 210 and data from the database 310.

In some embodiments, process 1100 further includes modeling the fourth value of the fluid property with the first value of the fluid property, the second value of the fluid property, and the third property of the fluid value to determine a sensor error. In some embodiments, the sensor error is associated with at least one of the first value of the fluid property, the second value of the fluid property, or the third value of the fluid property. In some embodiments, the sensor error may be determined by comparing trends in the sensor data over time. In various embodiments, the process 1100 may determine the sensor error similar to the process 1000 discussed herein.

In some embodiments, the process 1100 further includes displaying the sensor error to the user. In some embodiments, the user interface 320 is operated to provide display data of the sensor error. The display data of the sensor error may include elements associated with the sensor error. In some embodiments, the elements associated with the sensor error may include an identifier indicated a source of the sensor error (e.g., the measurement of the first capacitance, etc.). In some embodiments, the sensor error is displayed to the user in response to the sensor error being above a predetermined error threshold. The predetermined error threshold may be configured in order to identify if the capacitive sensor requires calibration. For example, the predetermined error threshold may be a value of the sensor error that corresponds to a requirement for calibration of the capacitive sensor.

In some embodiments, the process 1100 further includes calibrating the capacitive sensor. In some embodiments, the capacitive sensor is calibrated in response to the sensor error being above the predetermined error threshold. The calibration of the capacitive sensor may decrease the sensor error such that the sensor error is below the predetermined error threshold. For example, the calibration of the capacitive sensor may include filtering the sensor data provided by the capacitive sensor in order to decrease the sensor error.

In some embodiments, after calibrating the capacitive sensor, the process 1100 further includes receiving sensor data from the capacitive sensor corresponding to a first calibrated capacitance through a second fluid parcel of the fluid measured at a fourth time and a second calibrated capacitance through the second fluid parcel of the fluid measured at a fifth time. In some embodiments, the sensor data is received from the capacitive sensor 210, the capacitive sensor 500, the capacitive sensor 600, and/or the capacitive sensor 700. In some embodiments, the first calibrated capacitance may be received from the first of the electrode pairs of the capacitive sensor and the second calibrated capacitance may be received from the second of the electrode pairs of the capacitive sensor.

In some embodiments, the process 1100 includes determining a first calibrated value of the fluid property of the fluid based on the first calibrated capacitance. In some embodiments, the fluid property is permittivity and the first calibrated value of the fluid property is a first calibrated permittivity of the fluid. In some embodiments, the first calibrated value of the fluid property is determined based on physical relationships of the capacitive sensor and/or known fluid properties that correspond to the first calibrated capacitance. In some embodiments, the first calibrated capacitance is determined by the pipeline manager 312 of the controller 300 utilizing the sensor data from the capacitive sensor 210 and data from the database 310.

In some embodiments, the process 1100 includes determining a second calibrated value of the fluid property of the fluid based on the second calibrated capacitance. In some embodiments, the fluid property is permittivity and the second calibrated value of the fluid property is a second calibrated permittivity of the fluid. In some embodiments, the second calibrated value of the fluid property is determined based on physical relationships of the capacitive sensor and/or known fluid properties that correspond to the second calibrated capacitance. In some embodiments, the second calibrated capacitance is determined by the pipeline manager 312 of the controller 300 utilizing the sensor data from the capacitive sensor 210 and data from the database 310.

In some embodiments, the process 1100 includes determining a calibrated velocity of the fluid based on the fourth time and the fifth time. In some embodiments, the calibrated velocity of the fluid may be determined using physical relationships of the calibration sensor. In some embodiments, the calibrated velocity is determined by the pipeline manager 312 of the controller 300 utilizing the sensor data from the capacitive sensor 210 and data from the database 310.

In some embodiments, the process 1100 includes displaying the calibrated velocity of the fluid to the user. In some embodiments, process 1100 includes operating the user interface 320 to provide display data of the calibrated velocity. The display data of the calibrated velocity may include elements associated with the calibrated velocity. In some embodiments, the elements associated with the calibrated velocity are displayed to the user. For example, the elements may include an illustration of the calibrated velocity of the fluid. In some embodiments, the calibrated velocity is displayed by the user interface 320.

### Configuration of Exemplary Embodiments

As utilized herein, the terms “approximately”, “about”, “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It should be noted that the term “exemplary” as used herein to describe various embodiments is intended to indicate that such embodiments are possible examples, representations, and/or illustrations of possible embodiments (and such term is not intended to connote that such embodiments are necessarily extraordinary or superlative examples).

The terms “coupled,” “connected,” and the like, as used herein, mean the joining of two members directly or indirectly to one another. Such joining may be stationary (e.g., permanent) or moveable (e.g., removable, releasable, etc.). Such joining may be achieved with the two members or the two members and any additional intermediate members being integrally formed as a single unitary body with one another or with the two members or the two members and any additional intermediate members being attached to one another.

References herein to the positions of elements (e.g., “top,” “bottom,” “above,” “below,” etc.) are merely used to describe the orientation of various elements in the figures. It should be noted that the orientation of various elements may differ according to other exemplary embodiments, and that such variations are intended to be encompassed by the present disclosure.

Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, Z, X and Y, X and Z, Y and Z, or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

It is important to note that the construction and arrangement of the elements of the systems and methods as shown in the exemplary embodiments are illustrative only. Although only a few embodiments of the present disclosure have been described in detail, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements. It should be noted that the elements and/or assemblies of the components described herein may be constructed from any of a wide variety of materials that provide sufficient strength or durability, in any of a wide variety of colors, textures, and combinations. Accordingly, all such modifications are intended to be included within the scope of the present inventions. Other substitutions, modifications, changes, and omissions may be made in the design, operating conditions, and arrangement of the preferred and other exemplary embodiments without departing from scope of the present disclosure or from the spirit of the appended claims.

What is claimed is:

1. A system comprising:
a capacitive sensor configured to measure a first capacitance along a first path through a fluid and a second capacitance along a second path through the fluid, wherein the first path is different from the second path; and
a processor configured to:
receive sensor data from the capacitive sensor corresponding to the first capacitance and the second capacitance;
determine a first value of a fluid property of the fluid based on the first capacitance;
determine a second value of the fluid property of the fluid based on the second capacitance; and
responsive to determining that a variation between the first value of the fluid property and the second value of the fluid property is above a predetermined threshold, operate a display device to provide the variation to a user.

2. The system of claim 1, wherein the capacitive sensor is further configured to measure a third capacitance along a third path through the fluid, wherein the third path is different from the first path and the second path; and
wherein the processor is further configured to:
receive the sensor data from the capacitive sensor corresponding to the third capacitance;
determine a third value of the fluid property of the fluid based on the third capacitance;
model the first value of the fluid property with the second value of the fluid property and the third value of the fluid property to determine a sensor error associated with at least one of the first value of the fluid property, the second value of the fluid property, or the third value of the fluid property; and
responsive to determining that the sensor error is above a predetermined error threshold, operate the display device to provide the sensor error to the user.

3. The system of claim 2, wherein responsive to determining that the sensor error is above the predetermined error threshold, the processor is further configured to calibrate the capacitive sensor to reduce the sensor error.

4. The system of claim 2, wherein:
the capacitive sensor comprises:
a first electrode;
a second electrode;
a third electrode; and
a fourth electrode;
the first path is between (i) the first electrode and the third electrode and (ii) the second electrode and the fourth electrode;
the second path is between the first electrode and the third electrode; and
the third path is between the second electrode and the fourth electrode.

5. The system of claim 4, wherein the capacitive sensor further comprises:
a shield positioned between (i) the first electrode and the third electrode and (ii) the second electrode and the fourth electrode, the shield configured to partially block electric flux between (i) the first electrode and the third electrode and (ii) the second electrode and the fourth electrode.

6. The system of claim 2, wherein the sensor error is associated with a deposit of a material on the capacitive sensor or an erosion of the capacitive sensor due to the fluid.

7. The system of claim 6, wherein the processor is further configured to:
determine a location of (i) the deposit of the material on the capacitive sensor or (ii) the erosion of the capacitive sensor based on the sensor error; and
operate the display device to provide the location of (i) the deposit of the material or (ii) the location of the erosion of the capacitive sensor to the user.

8. The system of claim 1, wherein the fluid property is a permittivity of the fluid.

9. The system of claim 8, wherein the processor is further configured to:
determine a water fraction of the fluid based on the permittivity of the fluid.

10. A system comprising:
a capacitive sensor comprising a plurality of electrode pairs, each of the electrode pairs configured to measure a capacitance through a fluid between a first electrode and a second electrode of each of the electrode pairs; and
a processor configured to:
receive sensor data from the capacitive sensor corresponding to a first capacitance through a fluid parcel of the fluid at a first time from a first of the electrode pairs and a second capacitance through the fluid parcel of the fluid at a second time from a second of the electrode pairs;
determine a first value of a fluid property of the fluid based on the first capacitance;
determine a second value of the fluid property of the fluid based on the second capacitance, the second value substantially equal to the first value;
determine a velocity of the fluid based on the first time and the second time; and
operate a display device to provide the velocity of the fluid to a user.

11. The system of claim 10, wherein the processor is further configured to:
determine a third time when the fluid parcel of the fluid will be positioned proximate a third of the electrode pairs of the capacitive sensor;
receive the sensor data from the capacitive sensor corresponding to a third capacitance through the fluid parcel of the fluid at the third time from a third of the electrode pairs;
determine a third value of the fluid property of the fluid based on the third capacitance; and
responsive to determining that a variation between the first value of the fluid property and the third value of the fluid property is above a predetermined threshold, operate the display device to provide the variation to the user.

12. The system of claim 11, wherein the processor is further configured to:
receive the sensor data from the capacitive sensor corresponding to a fourth capacitance through the fluid parcel from the first electrode of the first of the electrode pairs and the first electrode of the second of the electrode pairs;
determine a fourth value of the fluid property of the fluid based on the fourth capacitance;
model the fourth value of the fluid property with the first value of the fluid property, the second value of the fluid property, and the third value of the fluid property to determine a sensor error associated with at least one of the first of the electrode pairs, the second of the electrode pairs, or the third of the electrode pairs; and
responsive to determining that the sensor error is above a predetermined error threshold, operate the display device to provide the sensor error to the user.

13. The system of claim 12, wherein responsive to determining that the sensor error is above the predetermined error threshold, the processor is further configured to calibrate the capacitive sensor to reduce the sensor error.

14. The system of claim 13, wherein:
the fluid parcel of the fluid is a first fluid parcel of the fluid;
the velocity is an initial velocity; and
the processor is further configured to:
receive the sensor data corresponding to a first calibrated capacitance through a second fluid parcel of the fluid measured at a fourth time from the first of the electrode pairs and a second calibrated capacitance through the second fluid parcel measured at a fifth time from the second of the electrode pairs;
determine a first calibrated value of the fluid property based on the first calibrated capacitance;
determine a second calibrated value of the fluid property based on the second calibrated capacitance, the second calibrated value substantially equal to the first calibrated value;
determine a calibrated velocity of the fluid based the fourth time and the fifth time; and
operate the display device to provide the calibrated velocity of the fluid to the user.

15. The system of claim 12, wherein:
the sensor error is associated with a deposit of a material on the capacitive sensor or an erosion of the capacitive sensor due to the fluid; and
the processor is further configured to:
determine a location of (i) the deposit of the material on the capacitive sensor or (ii) of the erosion of the capacitive sensor based on the sensor error; and
operate the display device to provide the location of (i) the deposit of the material or (ii) the erosion of the capacitive sensor to the user.

16. The system of claim 10, wherein the fluid property is a permittivity of the fluid.

17. The system of claim 16, wherein the processor is further configured to:
determine a water fraction of the fluid based on the permittivity of the fluid.

18. A method comprising:
receiving sensor data from a capacitive sensor corresponding to a first capacitance through a fluid along a first path and a second capacitance through the fluid along a second path;
determining a first value of a fluid property of the fluid based on the first capacitance;
determining a second value of the fluid property of the fluid based on the second capacitance; and
responsive to determining that a variation between the first value of the fluid property and the second value of the fluid property is above a predetermined threshold, operating a display device to provide the variation to a user.

19. The method of claim 18, further comprising:
receiving the sensor data from the capacitive sensor corresponding to a third capacitance through the fluid along a third path;
determining a third value of the fluid property of the fluid based on the third capacitance;
modeling the first value of the fluid property with the second value of the fluid property and the third value of the fluid property to determine a sensor error associated with the capacitive sensor and at least one of the first value of the fluid property, the second value of the fluid property, or the third value of the fluid property; and
responsive to determining that the sensor error is above a predetermined error threshold, operating the display device to provide the sensor error to the user.

20. The method of claim 19, wherein:
the fluid property is a permittivity of the fluid; and
the method further comprises:
determining a water fraction of the fluid based on the permittivity of the fluid.
